SEQUENCE LISTING (3 sequences)

SEQ ID NO: 1 - DNA, 4439 bp, Oryza sativa
ctaggacgatggtgtgatgtgggaacacgaagaaaacatgaggaaaaaatattaaaatgaatttcccacttaaaatgcatcaaataaaaaaaataaagaaacgaccgggaatagacacagggtttgtgaactagctagggcaaacatcatatggtcccttgctgatgcacaagtacattgagatgtcatttcaattctgtgcatcatatgcatgtggtcccttgctgaatattactcttgaaatatctaccagtgccaatctattgcatgacttaattaattcacaggttttgttgattacattattagtaagcttgagagcacaagctcaatggatttttctataaatggggatcattttgcaattttctttgtcgtgcaaagttagccttctttattactacttctgtttttaaatatacgatcctattgacttttggtcatatatttaaccatgtatcttatttagatagtttgcgcaaatatatataccttcaatgataaaattagttacaatgaaacaaatgatatttacgcaattctttttactaaacaagtcacaagaagtacctgcagcaatatatgttggaaccgtgcagtagatcgagcctagctacgcaaaaaaacaaaaagagaaaaaaagggaaaggaaaaacattaatcatgcatgagcagtatgcccggcaactggaatttgtcaaagatatggggagaggagaataatacaagtactactactacctagctctaccatgcatatgcacccaaaggcaaactggattattggataaagcacagatgctggcaaaacaatccttaagcctcccctccctgcttctttatttttgggcagcctctaccggacggtgccgtggtccattggaccagtaggtggcgacatacatggtttgggttaagtctaggagagcagtgtgtgtgcgcgcgcaagagagagagactgtgagtctgggagtagccctctcccctcctttggccatcttcctcgtgtatatgcatatatgcatcatcgcaacggtgtatatttgtggtgtggcgggtgtggcattggattgcccccattttggctcgtgcttcccagttagggtaaaacctgtggtaaacttgctagccccacgccaaagttacccttctttattgttgaaagggagaggaggtgtgtgaattgtgatggagggagagagagagagatagaaagagagatgtgtgtcaaagcaagcaagaaaccagtttcacaaagagctactactagtactagtgtactactgtggtacagtgcccaatgtcctttctccggactcgactccactaatattctcctcttctcgcgcggctcgttatattctcgtcatcattggaggctttagcaagcaagaagagaggcagtggtggtggtggtggaggaggagctagctagcctgtgcttgctgatcggtgctgagctgaggaatcgttcgatcgatcgggcgagtcgacgaggggaagagttgagctgaggcgcatcgagaacaagatcaacaggcaggtcaccttctccaagcgccgcaacggcctcctcaagaaggcctacgagctgtccgttctctgcgacgccgaggtcgcgctcatcatcttctccagccgcggcaagctctacgagttcggcagcgccgggtataattaatacagacacaacaacacacacaaccaacaaaccagcatcaatttgaacctgcagatctgctgttttctctgatcaattgcttctttttttttgttcttttttgtttcttttatctgctgcaacggcgtcctgctcctctggggtttctcgttttctttttcatttatttttagcaggtgccaagtagccgagctactatacttacctggccatgttaattattttattccgtctgtctgtgtgtgtctgtgcatactactatagggacatggcgcggtgttcttataaaccgggaggccggatccctaactagcatgggaggatatcttttcagcggatctatacaaaccctactcctgctgacctctttcttccagtttctccgggtcttccttggattattattgcccatcttccgggttgtgcgtgtgtcagagacagctcgaacgataaatttctcaaaaccagtactagagagggtgtgttgtgtgtgagaactgagtggagagttagcatgaaggctgcaaactagaaaggaaggtatgttctttcctttttgatccatcaggggagccccttctggtattaagatctttccggcacattgattttcatactttgtgatgaccctggaagaatcggcgtagcagcgtagcaccgctccattttggtcttaccctcacctccccatgctatgaactgatcaatttcattgttcttcatcacccttctcctagctttccacttccttcggatctcatgccatgtttctcagcatgaatcaaatttaattcgtgttttctacttccatatatactggaagaaatttaattagatctatttttgctcgggaggtcttcatactttgagttctgatgccatcaccttatttcccccccccccttctcttgttctatcttcttcctcatcttggcttgatcattttgatctgtcagttatagcatgatgcattctcaatttgactgtatgtaagttcaaccggaaatatgttgaatggattttctatatatcaacacttgatgtcaggcctgcatctgtttcgcttgtggtggtgtggccaaaattgtctatatttgatctttgctcttctttctcctcatttcatgacgattcctactacggcttaaaccattctttattctttactaatcatggatgttgcttgactcctagttgtttcgtactagctcaacttggagatcttttcattatttgcctagttggtgggtacgtttgtgacagatctaaaatggtgcacgaaaagttttacttattatgaaaaaagggagcttaacagggtaatttctctatttattcgtgatgacattttttccttgataagggggattttttataatctgcactcacatgtttatatgtaaaatctagctcttttgttttgtttttggcatatttcccgctaagtatagagtttatgtggataacattataacttttcaagatccaatccacatctttgattgtgaaaatcatacaatagggaaaatcaactgaagggttaattagatgctatatgcatatatatatatatgtgcgcgcgcgcgcgcctgaatttaactatgtatgcatccaactgtttcattgaaaaagatttgatatttttcagtctattctttttcgagtatatatttaatatgtttcaatctgttttgaccattataagataaagcctatattcaccaggcatttgagatgatcttttcatgcatgaaaaagctgttgttatcacttcaactaaccagacgatctaacatgtatttgtataagaaacagaccttgatttccttctgtaaaatcatgcatgtgttcgttttgaattggagtcggcgcgcctgtgttttgaccgtcaggaaagtcttttttttccctgaatagtcaagggtctatacttcttgaagcaattgggacactaatcaattattgtttatacctcggaccatcttttccttcttcacaccactaatcagtttatgccttggaccattaattgtgttgttcacaagcttcttgtttatggtttacaaagcattcgcctagatttgtgtgtgtctctacacatcgatcacttttaaatacttgtcgctttcagttattcttttaacgtttggttatttatcttatttaaaaaaattatcgtattattatttattttgtttgtgatttactttattatcaaaagtatttcaaatatgacttatctttttttataagtgcactaatttttcaaataagatgaatggtcaaatgttacaagaaaaagttaaagcaaccactaatttagggcggaggtagtaaaacctagttattgtaaccaataattttatcaatctataaatgcaacacaaagtcacttcgtgatatctcacacaaagccacttcaacgatgaaagctgactgcatgttttatcaaaacacatgtgatcagtttgttggatgaaaaaaattatctatgtcataaatcaagagttataatataagcttctggctctacaagtaacatttctatgttttttttttacgttcttacatactatgttttgccaaaaaaaacatgatcattttgttggacgaaaagaaatagtaaatatagagtgacctttgatatcattataatataagcttctgcctctataaataacatctatgcactttttacgtcgtagtaatttgatatatgagaaatttacatataacatttttgtgcagcataaccacc

SEQ ID NO: 2 - DNA, 411 bp, Zea mays
atggggagcctggcgtccgagaggaaggtggtcgggtgggccgccagggacgccaccggacacctctccccctactcctacaccctcaggaacacaggccctgaagatgtggtggtgaaggtgctctactgcgggatctgccacacggacatccaccaggccaagaaccacctcggggcttcaaagtatcctatggtccctgggcacgaggtggtcggcgaggtggtggaggtcgggcccgaggtggccaagtacggcgtcggcgacgtggtaggcgtcggggtgatcgttgggtgctgccgcgagtgcagcccctgcaaggccaacgttgagcagtactgcaacaagaagatctggtcatacaacgacgtctacactgatggacggcccacgcagggtggattcgccctc

SEQ ID NO: 3 - DNA, 363 bp, Zea mays
atgcagctggcgtcgtcgtccatcctgcccatgacgctgaagaacgccatcgagctgggcctgctggaggtgctgcagaaggaggccggcggcggcaaggcggcgctggcgcccgaggaggtggtggcgcggatgcccgcggcgcccggcgaccccgccgccgcggcggccatggtggaccgcatgctccgcctgctcgcctcctacgacgtcgtccggtgccagatggaggaccgggacggccggtacgagcgccgctactccgccgcgcccgtctgcaagtggctcacccccaacgaggacggcgtgtccatggccgccctcgcgctcatgaaccaggacaaggtcctcatggagagctgg

(19) United States
(12) Patent Application Publication
(10) Pub. No.: US 2006/0260011 A1
(43) Pub. Date: Nov. 16, 2006

(54) Methods and genetic constructs for modification of lignin composition of corn cobs

(75) Inventors: Royston E. Carter, Durham, NC (US); John Steffens, Research Triangle Park, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Participations AG, Basel (CH)

(21) Appl. No.: 11/388,275

(22) Filed: Mar. 24, 2006

Related U.S. Application Data
(60) Provisional application No. 60/665,695, filed on Mar. 28, 2005.

(51) Int. Cl. A01H 5/00; C12P 7/06; C12N 15/82

(52) U.S. Cl. 800/285; 435/161; 800/320.1

(57) ABSTRACT

The present invention relates to methods and genetic constructs for the control of expression of enzymes involved in lignin biosynthesis in plants. The method involves the use of double-stranded RNAi to down-regulate or knock out the expression of the CAD and COMT genes. In particular embodiments the method involves the use of cob-specific or cob-preferred promoters for down-regulation of lignin biosynthesis in the cobs of corn plants.

### CLAIM OF PRIORITY

This application claims priority to U.S. application Ser. No. 60/665,685, filed Mar. 28, 2005, the disclosure of which is hereby incorporated by reference.

### FIELD OF THE INVENTION

The present invention relates to the field of agricultural biotechnology. More specifically, the present invention relates to the use of RNAi technology to modify the expression of genes involved in the biosynthesis of lignin in maize (corn), and more specifically in maize cobs.

### BACKGROUND OF THE INVENTION

Lignin is a complex heterogeneous aromatic polymer which renders membranes impermeable and reinforces the walls of certain plants cells.

Lignin is formed by polymerization of free radicals derived from monolignols, such as paracoumaryl, coniferyl and sinapyl alcohols (Higuchi, 1985, in Biosynthesis and degradation of wood components (T. Higuchi, ed.), Academic Press, Orlando, Fla. pp. 141-160). Lignin is formed by polymerization of at least three different monolignols which are synthesized in a multistep pathway, each step in the pathway being catalyzed by a different enzyme. It has been shown that manipulation of the number of copies of genes encoding certain enzymes, such as cinnamyl alcohol dehydrogenase (CAD) and caffeic acid 3-O-methyltransferase (COMT) results in modification of the amount of lignin produced; see, for example, U.S. Pat. No. 5,451,514 and PCT publication no. WO 94/23044. Furthermore, it has been shown that antisense expression of sequences encoding CAD in poplar leads to the production of lignin having a modified composition (Grand, C. et al. Planta (Berl.) 163:232-237 (1985)).

Lignins have a wide variation in their relative content of monolignols, as a function of the species and the various tissues within the same plant

This variation is probably caused and controlled by different activities and specificities of substrates, the enzymes necessary for biosynthesis of lignin monomers (Higuchi, 1985, loc. cit.).

Beyond its role in the structure and development of plants, lignin represents a major component of the terrestrial biomass and assumes a major economic and ecological significance (Brown, 1985, J. Appl. Biochem. 7, 371-387; Whetten and Sederoff, 1991, Forest Ecology and Management, 43, 301-316).

At the level of exploitation of the biomass, it is appropriate first to note that lignin is a limiting factor of the digestibility and nutritional yield of fodder plants. In fact, it is clearly demonstrated that the digestibility of fodder plants by ruminants is inversely proportional to the content of lignin in these plants, the nature of the lignins also being a determining factor in this phenomenon (Buxton and Roussel, 1988, Crop. Sci., 28, 553-558; Jung and Vogel, 1986, J. Anim., Sci., 62, 1703-1712).

Among the main fodder plants in which it would be of interest to reduce the lignin contents there may be mentioned: lucerne, fescue and maize fodder used for silaging.

It should also be noted that high lignin contents are partly responsible for the limited quality of sunflower cake intended for feeding cattle, and for the reduction in germinative capacities of certain seeds in the horticultural sector.

It may also be emphasized that the intense lignification which results during preservation of plant components after harvesting rapidly renders products such as asparagus, yam, carrots etc, unfit for consumption.

Furthermore, it is also appropriate to note that more than 50 million tons of lignin are extracted from ligneous material each year in the context of production of paper pulp in the paper industry. This extraction operation, which is necessary to obtain cellulose, is costly in energy and, secondly, causes pollution through the chemical compounds used for the extraction, which are found in the environment (Dean and Eriksson, 1992, Holzforschung, 46, 135-147: Whetten and Sederoff, 1991, loc. cit.).

To reduce the proportions of lignins (which make up to 20 to 30% of the dry matter, depending on the species) to a few percent (2 to 5%) would represent an increase in yield and a substantial savings (chemical products), and would contribute to improving the environment (reduction in pollution). Given the scale of use of ligneous material, these decreases would have extremely significant repercussions. In this case, the species concerned could be poplar, eucalyptus, Acacia magnium, the genus Casuarina and all the angiosperms and gymnosperms used for the production of paper pulp.

It is clear that in the two sectors under consideration, the reduction in the levels of lignins must be moderated to preserve the characteristics of rigidity and the normal architecture of the plant (or the tree), since the lignins which strengthen the cell walls play a significant role in maintaining the erect habit of plants.

The natural variations in the lignin contents observed in nature for the same species (deviations which can be up to 6-8% of the dry matter among individuals) justify the reductions suggested above.

The resistance to degradation of lignin, like the difficulties encountered in the context of its extraction, are probably due to the complex structure of this polymer, which is made up of ether bonds and carbon-carbon bonds between the monomers, as well as to the numerous chemical bonds which exist between the lignin and the other components of the cell wall (Sarkanen and Ludwig, 1971, in Lignins: Occurrence, Formation, Structure and Reactions (K. V. Sarkanen and C. H. Kudwig ed.) New York: Wiley—Interscience, pp. 1-18).

An approach to attempt to reduce the level of lignins in plants by genetic engineering would consist of inhibiting the synthesis of one of the enzymes in the biosynthesis chain of these lignins indicated above.

A particularly suitable technique in the context of such an approach is to use antisense mRNA which is capable of hybridizing with the mRNA which codes for these enzymes, and consequently to prevent, at least partly, the production of these enzymes from their corresponding mRNA.

Such an antisense strategy carried out with the aid of the gene which codes for the CAD in tobacco was the subject matter of European Patent Application no. 584 117, which describes the use of antisense mRNA which is capable of inhibiting the production of lignins in plants by hybridizing with the mRNA which codes for the CAD in these plants.

The results in the plants transformed in this way demonstrate a reduction in the activity of the CAD, but paradoxically the contents of lignins show no change. Complementary studies indicate that the lignins of transformed plants are different from control lignins, since the cinnamylaldehydes are incorporated directly into the lignin polymer.

Brown mid rib (Bmr) corn has been used as an alternative for improving digestibility for silage hybrids for decades. The improvement in ruminal intakes and digestibility is derived from reduced lignin content in Bmr mutated hybrids. The Bm1 mutation is relatively mild and causes the fewest pleiotrophic effects, but it provides less digestibility improvement than Bm3, has been studied less, and has not been developed commercially. The Bm3 mutation is the best-studied Bm trait, it provides superior digestibility characteristics, but at the expense of moderately poor agronomic performance. Bm3 is the basis of existing commercial products. The Bm1 trait is caused by reduced activity of the biosynthetic enzyme, CAD and the Bm3 trait is caused by reduced activity of a biosynthetic enzyme, COMT.

The following background references are hereby incorporated herein by reference: U.S. Pat. Nos. 6,441,272; 6,855,864; 6,610,908; 5,451,514; 5,866,791; 5,959,178; 6,066,780; 6,211,432; 5,981,837; 5,850,020; 6,204,434; and 6,610,521; U.S. patent applications 20020081693 and 20030159170; PCT applications WO2004080202; WO03054229; European Application EP1425401; and Piquemal et al., Plant Physiology 130:1675-1685 (2002); Vignols et al., The Plant Cell 7:407-416 (1995); Morrow et al., Molecular Breeding 3:351-357 (1997). These references discuss various aspects of lignin biosynthesis in plants, and the control thereof. In particular, U.S. Pat. Nos. 5,451,514; 5,959,178; and 6,066,780 are particularly important with regard to their teaching regarding the role of CAD and COMT expression in lignin biosynthesis in plants.

Plant cells and tissues can respond to mechanical, chemical or pathogen induced injury by producing various phenolic compounds including mono- or dimethoxylated lignin precursors derived from cinnamic acid via a complex series of biochemical reactions. These lignin precursors are eventually used by the plant to produce the lignin polymer which helps in wound repair by adding hydrophobicity, a physical barrier against pathogen infection and mechanical strength to the injured tissue (Vance, C. P., et al., 1980, Annu Rev Phytopathol 18:259-288). Biosynthesis of the mono- or dimethoxylated lignin precursors occurs, in part, by the action of two enzymes, caffeic acid 3-O-methyltransferase (COMT), also known as caffeic acid/5-hydroxyferulic acid O-methyltransferase and caffeoyl CoA 3-O-methyltransferase (CCOMT). Both enzymes have been isolated and purified from a wide variety of plant species.

Studies have shown that the activities of COMT and CCOMT increase prior to lignin deposition (Inoue, K., et al., 1998, Plant Physiol 117(3):761-770). Synthesis of lignin precursors involves the methylation of caffeic acid to yield ferulic acid followed by 5-hydroxylation of ferulate then a second methyltion to yield sinapate. COMT has been implicated in the methylation of both caffeic acid and 5-hydroxyferulic acid ((Inoue, K., et al., 1998, Plant Physiol 117(3):761-770). Research indicates that COMT transcripts are present at high levels in organs containing vascular tissue and one study suggests that antisense inhibition of COMT can lead to modified lignin content and composition in the xylem and phloem of transgenic plant tissue (Dwivedi, U., et al., 1994, Plant Mol. Biol. 26:61-71).

A promising technology for achieving targeted gene silencing is based on double-stranded RNA (dsRNA) inducing a response called post-transcriptional gene silencing or RNA interference (RNAi). Double-stranded RNA has been introduced into a number of different species, including nematodes, fruit flies, Trypanosoma, fungi, plants. See for example, WO9932619. Some limited success has also been demonstrated in mammals, specifically in mouse oocytes and embryos. Introduction of the appropriate dsRNA inhibits gene expression in a sequence-dependent manner, an effect that has been used extensively in C. elegans and D. melanogaster as a genetic tool for studying gene function. For example, 00/01846 describes methods for characterizing gene function using dsRNA inhibition. However, dsRNA inhibition has been applied with little success in mammalian systems.

Because of the importance of lignins in cell wall architecture and digestibility, and because of the unfavourable agronomics of Bmr corn, there is considerable interest in the prospects for altering lignin quantity or quality by genetic engineering. Thus, there is a great deal of interest in identifying the genes that encode proteins involved in the production of lignin in plants and in modification of the expression of such genes, for example by the use of RNAi methods. These methods may be used in plant cells to control lignin production. Such methods would have significant utility in the production of plant material with improved digestibility, and if directed at decreasing lignin content of corn cobs, could avoid the agronomic downsides of the Bmr phenotype.

### SUMMARY OF THE INVENTION

The present invention provides methods and genetic expression constructs useful in the control of lignin biosynthesis in plants, and particularly in corn, and more particularly in the cobs of corn plants. In a specific example, double strand RNAi technology is utilized to decrease the expression of (or to knock out) either the cinnamyl-alcohol dehydrogenase (CAD) genes of maize or the caffeic acid O-methyl transferase (COMT) genes of maize. Preferred embodiments involve the knock out CAD or COMT genes specifically in the maize cob to reduce lignin content. This will provide improved digestibility of non-digestible fiber in the cob, which would improve whole plant digestibility by ruminants. Limiting expression of Bm-like traits only to the cob, the most highly lignified tissue, will still provide attractive increase in total plant digestibility, but mitigate most of the risk associated with poor agronomic performance such as increased lodging and poor dry-matter yield, as occurs with Bmr mutations, which is associated with their systemic expression. Therefore, CAD or COMT knock out events were generated using double strand RNAi technology with OsMAD6, a cob specific promoter.

The present invention relates to a method for controlling lignin biosynthesis in a plant, the method comprising down-regulating the expression of an enzyme in the plant, the enzyme selected from the group consisting of CAD and COMT, wherein the down-regulation is achieved using double-stranded RNAi. The method also relates to down-regulation of expression of both enzymes; to the dsRNAi constructs; and to cob-specific/cob-preferred constructs. The present invention also relates to the use of the low-lignin cobs produced using the method of the invention in biomass conversion applications (for example, in ethanol production) and in feed applications (for example, in animal feed for increased milk production, particularly in dairy cows).

### BRIEF DESCRIPTION OF THE DRAWINGS

FIG. 1 is a representation of plasmid pSyn12210

FIG. 2 is a representation of plasmid pSyn12345

FIG. 3 is a graph showing that as lignin content increases, digestibility of cob material decreases.

### DETAILED DESCRIPTION

Definitions

For clarity, certain terms used in the specification are defined and presented as follows:

“Associated with/operatively linked” refer to two nucleic acid sequences that are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

A “chimeric construct” is a recombinant nucleic acid sequence in which a promoter or regulatory nucleic acid sequence is operatively linked to, or associated with, a nucleic acid sequence that codes for an mRNA or which is expressed as a protein, such that the regulatory nucleic acid sequence is able to regulate transcription or expression of the associated nucleic acid sequence. The regulatory nucleic acid sequence of the chimeric construct is not normally operatively linked to the associated nucleic acid sequence as found in nature.

Co-factor: natural reactant, such as an organic molecule or a metal ion, required in an enzyme-catalyzed reaction. A co-factor is e.g. NAD(P), riboflavin (including FAD and FMN), folate, molybdopterin, thiamin, biotin, lipoic acid, pantothenic acid and coenzyme A, S-adenosylmethionine, pyridoxal phosphate, ubiquinone, menaquinone. Optionally, a co-factor can be regenerated and reused.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

Complementary: “complementary” refers to two nucleotide sequences that comprise antiparallel nucleotide sequences capable of pairing with one another upon formation of hydrogen bonds between the complementary base residues in the antiparallel nucleotide sequences.

Enzyme activity: means herein the ability of an enzyme to catalyze the conversion of a substrate into a product. A substrate for the enzyme comprises the natural substrate of the enzyme but also comprises analogues of the natural substrate, which can also be converted, by the enzyme into a product or into an analogue of a product. The activity of the enzyme is measured for example by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g. ATP, phosphoenolpyruvate, acetyl phosphate or phosphocreatine) remaining in the reaction mixture after a certain period of time or by determining the amount of a used donor of free energy or energy-rich molecule (e.g. ADP, pyruvate, acetate or creatine) in the reaction mixture after a certain period of time.

Expression Cassette: “Expression cassette” as used herein means a nucleic acid molecule capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue or organ or stage of development.

Gene: the term “gene” is used broadly to refer to any segment of DNA associated with a biological function. Thus, genes include coding sequences and/or the regulatory sequences required for their expression. Genes also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

Heterologous/exogenous: The terms “heterologous” and “exogenous” when used herein to refer to a nucleic acid sequence (e.g. a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” nucleic acid (e.g. DNA) sequence is a nucleic acid (e.g. DNA) sequence naturally associated with a host cell into which it is introduced.

Hybridization: The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

Inhibitor: a chemical substance that inactivates the enzymatic activity of a protein such as a biosynthetic enzyme, receptor, signal transduction protein, structural gene product, or transport protein. The term “herbicide” (or “herbicidal compound” is used herein to define an inhibitor applied to a plant at any stage of development, whereby the herbicide inhibits the growth of the plant or kills the plant.

Interaction: quality or state of mutual action such that the effectiveness or toxicity of one protein or compound on another protein is inhibitory (antagonists) or enhancing (agonists).

A nucleic acid sequence is “isocoding with” a reference nucleic acid sequence when the nucleic acid sequence encodes a polypeptide having the same amino acid sequence as the polypeptide encoded by the reference nucleic acid sequence.

Isogenic: plants that are genetically identical, except that they may differ by the presence or absence of a heterologous DNA sequence.

Isolated: in the context of the present invention, an isolated DNA molecule or an isolated enzyme is a DNA molecule or enzyme that, by the hand of man, exists apart from its native environment and is therefore not a product of nature. An isolated DNA molecule or enzyme may exist in a purified form or may exist in a non-native environment such as, for example, in a transgenic host cell.

Mature protein: protein from which the transit peptide, signal peptide, and/or propeptide portions have been removed.

Minimal Promoter the smallest piece of a promoter, such as a TATA element, that can support any transcription. A minimal promoter typically has greatly reduced promoter activity in the absence of upstream activation. In the presence of a suitable transcription factor, the minimal promoter functions to permit transcription.

Modified Enzyme Activity: enzyme activity different from that which naturally occurs in a plant (i.e. enzyme activity that occurs naturally in the absence of direct or indirect manipulation of such activity by man), which is tolerant to inhibitors that inhibit the naturally occurring enzyme activity.

Native: refers to a gene that is present in the genome of an untransformed plant cell.

Naturally occurring: the term “naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

Nucleic acid: the term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al, Nucleic Acid Res. 19: 5081 (1991); Ohtsuka et al., J. Biol. Chem. 260: 2605-2608 (1985); Rossolini et al., Mol. Cell. Probes 8: 91-98 (1994)). The terms “nucleic acid” or “nucleic acid sequence” may also be used interchangeably with gene, cDNA, and mRNA encoded by a gene.

“ORF” means open reading frame.

Percent identity: the phrases “percent identical” or “percent identical,” in the context of two nucleic acid or protein sequences, refers to two or more sequences or subsequences that have for example 60%, preferably 70%, more preferably 80%, still more preferably 90%, even more preferably 95%, and most preferably at least 99% nucleotide or amino acid residue identity, when compared and aligned for maximum correspondence, as measured using one of the following sequence comparison algorithms or by visual inspection. Preferably, the percent identity exists over a region of the sequences that is at least about 50 residues in length, more preferably over a region of at least about 100 residues, and most preferably the percent identity exists over at least about 150 residues. In an especially preferred embodiment, the percent identity exists over the entire length of the coding regions.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.go-v/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Pre-protein: protein that is normally targeted to a cellular organelle, such as a chloroplast, and still comprises its native transit peptide.

Purified: the term “purified,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least about 50% pure, more preferably at least about 85% pure, and most preferably at least about 99% pure.

Two nucleic acids are “recombined” when sequences from each of the two nucleic acids are combined in a progeny nucleic acid. Two sequences are “directly” recombined when both of the nucleic acids are substrates for recombination. Two sequences are “indirectly recombined” when the sequences are recombined using an intermediate such as a cross-over oligonucleotide. For indirect recombination, no more than one of the sequences is an actual substrate for recombination, and in some cases, neither sequence is a substrate for recombination.

“Regulatory elements” refer to sequences involved in controlling the expression of a nucleotide sequence. Regulatory elements comprise a promoter operatively linked to the nucleotide sequence of interest and termination signals. They also typically encompass sequences required for proper translation of the nucleotide sequence.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

Significantly less: means that the amount of a product of an enzymatic reaction is reduced by more than the margin of error inherent in the measurement technique, preferably a decrease by about 2-fold or greater of the activity of the wild-type enzyme in the absence of the inhibitor, more preferably an decrease by about 5-fold or greater, and most preferably an decrease by about 10-fold or greater.

Specific Binding/Immunological Cross-Reactivity: An indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions. The phrase “specifically (or selectively) binds to an antibody,” or “specifically (or selectively) immunoreactive with,” when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised to the protein with the amino acid sequence encoded by any of the nucleic acid sequences of the invention can be selected to obtain antibodies specifically immunoreactive with that protein and not with other proteins except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See Harlow and Lane (1988) Antibodies, A Laboratory Manual, Cold Spring Harbor Publications, New York “Harlow and Lane”), for a description of immunoassay formats and conditions that can be used to determine specific immunoreactivity. Typically a specific or selective reaction will be at least twice background signal or noise and more typically more than 10 to 100 times background.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5.degree. C. lower than the thermal melting point (T.sub.m) for the specific sequence at a defined ionic strength and pH. Typically, under “stringent conditions” a probe will hybridize to its target subsequence, but to no other sequences.

The T.sub.m is the temperature (under defined ionic strength and pH) at which 50% of the target sequence hybridizes to a perfectly matched probe. Very stringent conditions are selected to be equal to the T.sub.m for a particular probe. An example of stringent hybridization conditions for hybridization of complementary nucleic acids which have more than 100 complementary residues on a filter in a Southern or northern blot is 50% formamide with 1 mg of heparin at 42.degree. C., with the hybridization being carried out overnight. An example of highly stringent wash conditions is 0.1 5M NaCl at 72.degree. C. for about 15 minutes. An example of stringent wash conditions is a 0.2.times.SSC wash at 65.degree. C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1.times.SSC at 45.degree. C. for 15 minutes. An example low stringency wash for a duplex of, e.g., more than 100 nucleotides, is 4-6.times.SSC at 40.degree. C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30.degree. C. Stringent conditions can also be achieved with the addition of destabilizing agents such as formamide. In general, a signal to noise ratio of 2.times (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences of the present invention: a reference nucleotide sequence preferably hybridizes to the reference nucleotide sequence in 7% sodium dodecyl sulfate (SDS), 0.5 M NaPO.sub.4, 1 mM EDTA at 50.degree. C. with washing in 2.times.SSC, 0.1% SDS at 50.degree. C., more desirably in 7% sodium dodecyl sulfate (SDS), 0.5 M NaPO.sub.4, 1 mM EDTA at 50.degree. C. with washing in 1.times.SSC, 0.1% SDS at 50.degree. C., more desirably still in 7% sodium dodecyl sulfate (SDS), 0.5 M NaPO.sub.4, 1 mM EDTA at 50.degree. C. with washing in 0.5.times.SSC, 0.1% SDS at 50.degree. C., preferably in 7% sodium dodecyl sulfate (SDS), 0.5 M NaPO.sub.4, 1 mM EDTA at 50.degree. C. with washing in 0.1.times.SSC, 0.1% SDS at 50.degree. C., more preferably in 7% sodium dodecyl sulfate (SDS), 0.5 M NaPO.sub.4, 1 mM EDTA at 50.degree. C. with washing in 0.1.times.SSC, 0.1% SDS at 65.degree. C.

A “subsequence” refers to a sequence of nucleic acids or amino acids that comprise a part of a longer sequence of nucleic acids or amino acids (e.g., protein) respectively.

Substrate: a substrate is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

Transformation: a process for introducing heterologous DNA into a plant cell, plant tissue, or plant. Transformed plant cells, plant tissue, or plants are understood to encompass not only the end product of a transformation process, but also transgenic progeny thereof.

“Transformed,” “transgenic,” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

Viability: “viability” as used herein refers to a fitness parameter of a plant. Plants are assayed for their homozygous performance of plant development, indicating which proteins are essential for plant growth. dsRNA

Alteration of the expression of a nucleotide sequence of the present invention is also obtained by dsRNA interference as described for example in WO 99/32619, WO 99/53050 or WO 99/61631, all incorporated herein by reference in their entirety. In another preferred embodiment, the alteration of the expression of a nucleotide sequence of the present invention, preferably the reduction of its expression, is obtained by double-stranded RNA (dsRNA) interference. The entirety or, preferably a portion of a nucleotide sequence of the present invention is comprised in a DNA molecule. The size of the DNA molecule is preferably from 100 to 1000 nucleotides or more; the optimal size to be determined empirically. Two copies of the identical DNA molecule are linked, separated by a spacer DNA molecule, such that the first and second copies are in opposite orientations. In the preferred embodiment, the first copy of the DNA molecule is in the reverse complement (also known as the non-coding strand) and the second copy is the coding strand; in the most preferred embodiment, the first copy is the coding strand, and the second copy is the reverse complement. The size of the spacer DNA molecule is preferably 200 to 10,000 nucleotides, more preferably 400 to 5000 nucleotides and most preferably 600 to 1500 nucleotides in length. The spacer is preferably a random piece of DNA, more preferably a random piece of DNA without homology to the target organism for dsRNA interference, and most preferably a functional intron which is effectively spliced by the target organism. The two copies of the DNA molecule separated by the spacer are operatively linked to a promoter functional in a plant cell, and introduced in a plant cell, in which the nucleotide sequence is expressible. In a preferred embodiment, the DNA molecule comprising the nucleotide sequence, or a portion thereof, is stably integrated in the genome of the plant cell.

In another preferred embodiment the DNA molecule comprising the nucleotide sequence, or a portion thereof, is comprised in an extrachromosomally replicating molecule. Several publications describing this approach are cited for further illustration (Waterhouse et al. (1998) PNAS 95:13959-13964; Chuang and Meyerowitz (2000) PNAS 97:49854990; Smith et al. (2000) Nature 407:319-320). Alteration of the expression of a nucleotide sequence by dsRNA interference is also described in, for example WO 99/32619, WO 99/53050 or WO 99/61631, all incorporated herein by reference in their entirety

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

Controlling Gene Expression in Transgenic Plants

The invention further relates to transformed cells comprising the nucleic acid molecules, transformed plants, seeds, and plant parts, and methods of modifying phenotypic traits of interest by altering the expression of the genes of the invention.

A. Modification of Coding Sequences and Adjacent Sequences

The transgenic expression in plants of genes derived from heterologous sources may involve the modification of those genes to achieve and optimize their expression in plants. In particular, bacterial ORFs which encode separate enzymes but which are encoded by the same transcript in the native microbe are best expressed in plants on separate transcripts. To achieve this, each microbial ORF is isolated individually and cloned within a cassette which provides a plant promoter sequence at the 5′ end of the ORF and a plant transcriptional terminator at the 3′ end of the ORF. The isolated ORF sequence preferably includes the initiating ATG codon and the terminating STOP codon but may include additional sequence beyond the initiating ATG and the STOP codon. In addition, the ORF may be truncated, but still retain the required activity; for particularly long ORFs, truncated versions which retain activity may be preferable for expression in transgenic organisms. By “plant promoter” and “plant transcriptional terminator” it is intended to mean promoters and transcriptional terminators which operate within plant cells. This includes promoters and transcription terminators which may be derived from non-plant sources such as viruses (an example is the Cauliflower Mosaic Virus).

In some cases, modification to the ORF coding sequences and adjacent sequence is not required. It is sufficient to isolate a fragment containing the ORF of interest and to insert it downstream of a plant promoter. For example, Gaffney et al. (Science 261: 754-756 (1993)) have expressed the Pseudomonas nahG gene in transgenic plants under the control of the CaMV ³⁵S promoter and the CaMV tml terminator successfully without modification of the coding sequence and with nucleotides of the Pseudomonas gene upstream of the ATG still attached, and nucleotides downstream of the STOP codon still attached to the nahG ORF. Preferably as little adjacent microbial sequence should be left attached upstream of the ATG and downstream of the STOP codon. In practice, such construction may depend on the availability of restriction sites.

In other cases, the expression of genes derived from microbial sources may provide problems in expression. These problems have been well characterized in the art and are particularly common with genes derived from certain sources such as Bacillus. These problems may apply to the nucleotide sequence of this invention and the modification of these genes can be undertaken using techniques now well known in the art. The following problems may be encountered:

1. Codon Usage.

The preferred codon usage in plants differs from the preferred codon usage in certain microorganisms. Comparison of the usage of codons within a cloned microbial ORF to usage in plant genes (and in particular genes from the target plant) will enable an identification of the codons within the ORF which should preferably be changed. Typically plant evolution has tended towards a strong preference of the nucleotides C and G in the third base position of monocotyledons, whereas dicotyledons often use the nucleotides A or T at this position. By modifying a gene to incorporate preferred codon usage for a particular target transgenic species, many of the problems described below for GC/AT content and illegitimate splicing will be overcome.

2. GC/AT Content.

Plant genes typically have a GC content of more than 35%. ORF sequences which are rich in A and T nucleotides can cause several problems in plants. Firstly, motifs of ATTTA are believed to cause destabilization of messages and are found at the 3′ end of many short-lived mRNAs. Secondly, the occurrence of polyadenylation signals such as AATAAA at inappropriate positions within the message is believed to cause premature truncation of transcription. In addition, monocotyledons may recognize AT-rich sequences as splice sites (see below).

3. Sequences Adjacent to the Initiating Methionine.

Plants differ from microorganisms in that their messages do not possess a defined ribosome binding site. Rather, it is believed that ribosomes attach to the 5′ end of the message and scan for the first available ATG at which to start translation. Nevertheless, it is believed that there is a preference for certain nucleotides adjacent to the ATG and that expression of microbial genes can be enhanced by the inclusion of a eukaryotic consensus translation initiator at the ATG. Clontech (1993/1994 catalog, page 210, incorporated herein by reference) have suggested one sequence as a consensus translation initiator for the expression of the E. coli uidA gene in plants. Further, Joshi (N.A.R. 15: 6643-6653 (1987), incorporated herein by reference) has compared many plant sequences adjacent to the ATG and suggests another consensus sequence. In situations where difficulties are encountered in the expression of microbial ORFs in plants, inclusion of one of these sequences at the initiating ATG may improve translation. In such cases the last three nucleotides of the consensus may not be appropriate for inclusion in the modified sequence due to their modification of the second AA residue. Preferred sequences adjacent to the initiating methionine may differ between different plant species. A survey of 14 maize genes located in the GenBank database provided the following results:

1 Position Before the Initiating ATG in 14 Maize Genes: −10 −9 −8 −7 −6 −5 −4 −3 −2 −1 C3 8 4 6 2 5 6 0 1 0 7T3 0 3 4 3 2 1 1 1 0A2 3 1 4 3 2 3 7 2 3 G6 3 6 0 6 5 4 6 1 5

This analysis can be done for the desired plant species into which the nucleotide sequence is being incorporated, and the sequence adjacent to the ATG modified to incorporate the preferred nucleotides.

4. Removal of Illegitimate Splice Sites.

Genes cloned from non-plant sources and not optimized for expression in plants may also contain motifs which may be recognized in plants as 5′ or 3′ splice sites, and be cleaved, thus generating truncated or deleted messages. These sites can be removed using the techniques well known in the art.

Techniques for the modification of coding sequences and adjacent sequences are well known in the art. In cases where the initial expression of a microbial ORF is low and it is deemed appropriate to make alterations to the sequence as described above, then the construction of synthetic genes can be accomplished according to methods well known in the art. These are, for example, described in the published patent disclosures EP 0 385 962 (to Monsanto), EP 0 359 472 (to Lubrizol) and WO 93/07278 (to Ciba-Geigy), all of which are incorporated herein by reference. In most cases it is preferable to assay the expression of gene constructions using transient assay protocols (which are well known in the art) prior to their transfer to transgenic plants.

B. Construction of Plant Expression Cassettes

Coding sequences intended for expression in transgenic plants are first assembled in expression cassettes behind a suitable promoter expressible in plants. The expression cassettes may also comprise any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors described below. The following is a description of various components of typical expression cassettes.

1. Promoters

The selection of the promoter used in expression cassettes will determine the spatial and temporal expression pattern of the transgene in the transgenic plant. Selected promoters will express transgenes in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the gene under various inducing conditions. Promoters vary in their strength, i.e., ability to promote transcription. Depending upon the host cell system utilized, any one of a number of suitable promoters can be used, including the gene's native promoter. The following are non-limiting examples of promoters that may be used in expression cassettes.

a. Constitutive Expression, the Ubiquitin Promoter:

Ubiquitin is a gene product known to accumulate in many cell types and its promoter has been cloned from several species for use in transgenic plants (e.g. sunflower—Binet et al. Plant Science 79: 87-94 (1991); maize—Christensen et al. Plant Molec.

Biol. 12: 619-632 (1989); and Arabidopsis—Callis et al., J. Biol. Chem. 265: 12486-12493 (1990) and Norris et al., Plant Mol. Biol. 21: 895-906 (1993)). The maize ubiquitin promoter has been developed in transgenic monocot systems and its sequence and vectors constructed for monocot transformation are disclosed in the patent publication EP 0 342 926 (to Lubrizol) which is herein incorporated by reference. Taylor et al. (Plant Cell Rep. 12: 491495 (1993)) describe a vector (pAHC25) that comprises the maize ubiquitin promoter and first intron and its high activity in cell suspensions of numerous monocotyledons when introduced via microprojectile bombardment. The Arabidopsis ubiquitin promoter is ideal for use with the nucleotide sequences of the present invention. The ubiquitin promoter is suitable for gene expression in transgenic plants, both monocotyledons and dicotyledons. Suitable vectors are derivatives of pAHC25 or any of the transformation vectors described in this application, modified by the introduction of the appropriate ubiquitin promoter and/or intron sequences.

b. Constitutive Expression, the CaMV 35S Promoter:

Construction of the plasmid pCGN 1761 is described in the published patent application EP 0 392 225 (Example 23), which is hereby incorporated by reference. pCGN1761 contains the “double” CaMV 35S promoter and the tml transcriptional terminator with a unique EcoRI site between the promoter and the terminator and has a pUC-type backbone. A derivative of pCGN1761 is constructed which has a modified polylinker which includes NotI and XhoI sites in addition to the existing EcoRI site. This derivative is designated pCGN1761ENX. pCGN1761ENX is useful for the cloning of cDNA sequences or coding sequences (including microbial ORF sequences) within its polylinker for the purpose of their expression under the control of the 35S promoter in transgenic plants. The entire 35S promoter-coding sequence-tml terminator cassette of such a construction can be excised by HindIII, SphI, SalI, and XbaI sites 5′ to the promoter and XbaI, BamHI and BglI sites 3′) to the terminator for transfer to transformation vectors such as those described below. Furthermore, the double 35S promoter fragment can be removed by 5′ excision with HindIII, SphI, SalI, XbaI, or PstI, and 3′ excision with any of the polylinker restriction sites (EcoRI, NotI or XhoI) for replacement with another promoter. If desired, modifications around the cloning sites can be made by the introduction of sequences that may enhance translation. This is particularly useful when overexpression is desired. For example, pCGN1761 ENX may be modified by optimization of the translational initiation site as described in Example 37 of U.S. Pat. No. 5,639,949, incorporated herein by reference.

c. Constitutive Expression, the Actin Promoter:

Several isoforms of actin are known to be expressed in most cell types and consequently the actin promoter is a good choice for a constitutive promoter. In particular, the promoter from the rice Actl gene has been cloned and characterized (McElroy et al. Plant Cell 2: 163-171 (1990)). A 1.3 kb fragment of the promoter was found to contain all the regulatory elements required for expression in rice protoplasts. Furthermore, numerous expression vectors based on the Actl promoter have been constructed specifically for use in monocotyledons (McElroy et al. Mol. Gen. Genet. 231: 150-160 (1991)). These incorporate the Actl-intron 1, Adhl 5′ flanking sequence and Adhl-intron 1 (from the maize alcohol dehydrogenase gene) and sequence from the CaMV 35S promoter. Vectors showing highest expression were fusions of 35S and Actl intron or the Actl 5′ flanking sequence and the Actl intron. Optimization of sequences around the initiating ATG (of the GUS reporter gene) also enhanced expression. The promoter expression cassettes described by McElroy et al. (Mol. Gen. Genet. 231: 150-160 (1991)) can be easily modified for gene expression and are particularly suitable for use in monocotyledonous hosts. For example, promoter-containing fragments is removed from the McElroy constructions and used to replace the double 35S promoter in pCGN1761ENX, which is then available for the insertion of specific gene sequences. The fusion genes thus constructed can then be transferred to appropriate transformation vectors. In a separate report, the rice Actl promoter with its first intron has also been found to direct high expression in cultured barley cells (Chibbar et al. Plant Cell Rep. 12: 506-509 (1993)).

d. Inducible Expression, PR-1 Promoters:

The double 35S promoter in pCGN1761 ENX may be replaced with any other promoter of choice that will result in suitably high expression levels. By way of example, one of the chemically regulatable promoters described in U.S. Pat. No. 5,614,395, such as the tobacco PR-1 promoter, may replace the double 35S promoter. Alternately, the Arabidopsis PR-1 promoter described in Lebel et al., Plant J. 16: 223-233 (1998) may be used. The promoter of choice is preferably excised from its source by restriction enzymes, but can alternatively be PCR-amplified using primers that carry appropriate terminal restriction sites. Should PCR-amplification be undertaken, then the promoter should be re-sequenced to check for amplification errors after the cloning of the amplified promoter in the target vector. The chemically/pathogen regulatable tobacco PR-1 a promoter is cleaved from plasmid pCIB 1004 (for construction, see example 21 of EP 0332 104, which is hereby incorporated by reference) and transferred to plasmid pCGN1761ENX (Uknes et al., Plant Cell 4: 645-656 (1992)). pCIB1004 is cleaved with NcoI and the resultant 3′ overhang of the linearized fragment is rendered blunt by treatment with T4 DNA polymerase. The fragment is then cleaved with HindIII and the resultant PR-1 a promoter-containing fragment is gel purified and cloned into pCGN 1761 ENX from which the double 35S promoter has been removed. This is done by cleavage with XhoI and blunting with T4 polymerase, followed by cleavage with HindIII and isolation of the larger vector-terminator containing fragment into which the pCIB 1004 promoter fragment is cloned. This generates a pCGN 1761 ENX derivative with the PR-1a promoter and the tml terminator and an intervening polylinker with unique EcoRI and NotI sites. The selected coding sequence can be inserted into this vector, and the fusion products (i.e. promoter-gene-terminator) can subsequently be transferred to any selected transformation vector, including those described infra. Various chemical regulators may be employed to induce expression of the selected coding sequence in the plants transformed according to the present invention, including the benzothiadiazole, isonicotinic acid, and salicylic acid compounds disclosed in U.S. Pat. Nos. 5,523,311 and 5,614,395.

e. Inducible Expression, an Ethanol-Inducible Promoter:

A promoter inducible by certain alcohols or ketones, such as ethanol, may also be used to confer inducible expression of a coding sequence of the present invention. Such a promoter is for example the alcA gene promoter from Aspergillus nidulans (Caddick et al. (1998) Nat. Biotechnol 16:177-180). In A. nidulans, the alcA gene encodes alcohol dehydrogenase 1, the expression of which is regulated by the AlcR transcription factors in presence of the chemical inducer. For the purposes of the present invention, the CAT coding sequences in plasmid palcA:CAT comprising a alcA gene promoter sequence fused to a minimal 35S promoter (Caddick et al. (1998) Nat. Biotechnol 16:177-180) are replaced by a coding sequence of the present invention to form an expression cassette having the coding sequence under the control of the alcA gene promoter. This is carried out using methods well known in the art.

f. Inducible Expression, a Glucocorticoid-Inducible Promoter:

Induction of expression of a nucleic acid sequence of the present invention using systems based on steroid hormones is also contemplated. For example, a glucocorticoid-mediated induction system is used (Aoyama and Chua (1997) The Plant Journal 11: 605-612) and gene expression is induced by application of a glucocorticoid, for example a synthetic glucocorticoid, preferably dexamethasone, preferably at a concentration ranging from 0.1 mM to 1 mM, more preferably from 10 mM to 100 mM. For the purposes of the present invention, the luciferase gene sequences are replaced by a nucleic acid sequence of the invention to form an expression cassette having a nucleic acid sequence of the invention under the control of six copies of the GAL4 upstream activating sequences fused to the 35S minimal promoter. This is carried out using methods well known in the art. The trans-acting factor comprises the GAL4 DNA-binding domain (Keegan et al. (1986) Science 231: 699-704) fused to the transactivating domain of the herpes viral protein VP 16 (Triezenberg et al. (1988) Genes Devel. 2: 718-729) fused to the hormone-binding domain of the rat glucocorticoid receptor (Picard et al. (1988) Cell 54: 1073-1080). The expression of the fusion protein is controlled by any promoter suitable for expression in plants known in the art or described here. This expression cassette is also comprised in the plant comprising a nucleic acid sequence of the invention fused to the 6.times.GAL4/minimal promoter. Thus, tissue- or organ-specificity of the fusion protein is achieved leading to inducible tissue- or organ-specificity of the insecticidal toxin.

g. Root Specific Expression:

Another pattern of gene expression is root expression. A suitable root promoter is the promoter of the maize metallothionein-like (MTL) gene described by de Framond (FEBS 290: 103-106 (1991)) and also in U.S. Pat. No. 5,466,785, incorporated herein by reference. This “MTL” promoter is transferred to a suitable vector such as pCGN1761 ENX for the insertion of a selected gene and subsequent transfer of the entire promoter-gene-terminator cassette to a transformation vector of interest.

h. Wound-Inducible Promoters:

Wound-inducible promoters may also be suitable for gene expression. Numerous such promoters have been described (e.g. Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), Warner et al. Plant J. 3: 191-201 (1993)) and all are suitable for use with the instant invention. Logemann et al. describe the 5′ upstream sequences of the dicotyledonous potato wunl gene. Xu et al. show that a wound-inducible promoter from the dicotyledon potato (pin2) is active in the monocotyledon rice. Further, Rohrmeier & Lehle describe the cloning of the maize Wipl cDNA which is wound induced and which can be used to isolate the cognate promoter using standard techniques. Similar, Firek et al. and Warner et al. have described a wound-induced gene from the monocotyledon Asparagus officinalis, which is expressed at local wound and pathogen invasion sites.

Using cloning techniques well known in the art, these promoters can be transferred to suitable vectors, fused to the genes pertaining to this invention, and used to express these genes at the sites of plant wounding.

i. Pith-Preferred Expression:

Patent Application WO 93/07278, which is herein incorporated by reference, describes the isolation of the maize trpA gene, which is preferentially expressed in pith cells. The gene sequence and promoter extending up to −1726 bp from the start of transcription are presented. Using standard molecular biological techniques, this promoter, or parts thereof, can be transferred to a vector such as pCGN 1761 where it can replace the 35S promoter and be used to drive the expression of a foreign gene in a pith-preferred manner. In fact, fragments containing the pith-preferred promoter or parts thereof can be transferred to any vector and modified for utility in transgenic plants.

j. Leaf-Specific Expression:

A maize gene encoding phosphoenol carboxylase (PEPC) has been described by Hudspeth & Grula (Plant Molec Biol 12: 579-589 (1989)). Using standard molecular biological techniques the promoter for this gene can be used to drive the expression of any gene in a leaf-specific manner in transgenic plants.

k. Pollen-Specific Expression:

WO 93/07278 describes the isolation of the maize calcium-dependent protein kinase (CDPK) gene which is expressed in pollen cells. The gene sequence and promoter extend up to 1400 bp from the start of transcription. Using standard molecular biological techniques, this promoter or parts thereof, can be transferred to a vector such as pCGN1761 where it can replace the 35S promoter and be used to drive the expression of a nucleic acid sequence of the invention in a pollen-specific manner.

l. Cob-Specific Expression:

The OsMAD6 promoter, isolated from rice, is in maize a cob specific promoter, having the sequence (SEQ ID NO: 1):

ctaggacgatggtgtgatgtgggaacacgaagaaaacatgaggaaaaaat
attaaaatgaatttcccacttaaaatgcatcaaataaaaaaaataaagaa
acgaccgggaatagacacagggtttgtgaactagctagggcaaacatcat
atggtcccttgctgatgcacaagtacattgagatgtcatttcaattctgt
gcatcatatgcatgtggtcccttgctgaatattactcttgaaatatctac
cagtgccaatctattgcatgacttaattaattcacaggttttgttgatta
cattattagtaagcttgagagcacaagctcaatggatttttctataaatg
gggatcattttgcaattttctttgtcgtgcaaagttagccttctttatta
ctacttctgtttttaaatatacgatcctattgacttttggtcatatattt
aaccatgtatcttatttagatagtttgcgcaaatatatataccttcaatg
ataaaattagttacaatgaaacaaatgatatttacgcaattctttttact
aaacaagtcacaagaagtacctgcagcaatatatgttggaaccgtgcagt
agatcgagcctagctacgcaaaaaaacaaaaagagaaaaaaagggaaagg
aaaaacattaatcatgcatgagcagtatgcccggcaactggaatttgtca
aagatatggggagaggagaataatacaagtactactactacctagctcta
ccatgcatatgcacccaaaggcaaactggattattggataaagcacagat
gctggcaaaacaatccttaagcctcccctccctgcttctttatttttggg
cagcctctaccggacggtgccgtggtccattggaccagtaggtggcgaca
tacatggtttgggttaagtctaggagagcagtgtgtgtgcgcgcgcaaga
gagagagactgtgagtctgggagtagccctctcccctcctttggccatct
tcctcgtgtatatgcatatatgcatcatcgcaacggtgtatatttgtggt
gtggcgggtgtggcattggattgcccccattttggctcgtgcttcccagt
tagggtaaaacctgtggtaaacttgctagccccacgccaaagttaccctt
ctttattgttgaaagggagaggaggtgtgtgaattgtgatggagggagag
agagagagatagaaagagagatgtgtgtcaaagcaagcaagaaaccagtt
tcacaaagagctactactagtactagtgtactactgtggtacagtgccca
atgtcctttctccggactcgactccactaatattctcctcttctcgcgcg
gctcgttatattctcgtcatcattggaggctttagcaagcaagaagagag
gcagtggtggtggtggtggaggaggagctagctagcctgtgcttgctgat
cggtgctgagctgaggaatcgttcgatcgatcgggcgagtcgacgagggg
aagagttgagctgaggcgcatcgagaacaagatcaacaggcaggtcacct
tctccaagcgccgcaacggcctcctcaagaaggcctacgagctgtccgtt
ctctgcgacgccgaggtcgcgctcatcatcttctccagccgcggcaagct
ctacgagttcggcagcgccgggtataattaatacagacacaacaacacac
acaaccaacaaaccagcatcaatttgaacctgcagatctgctgttttctc
tgatcaattgcttctttttttttgttcttttttgtttcttttatctgctg
caacggcgtcctgctcctctggggtttctcgttttctttttcatttattt
ttagcaggtgccaagtagccgagctactatacttacctggccatgttaat
tattttattccgtctgtctgtgtgtgtctgtgcatactactatagggaca
tggcgcggtgttcttataaaccgggaggccggatccctaactagcatggg
aggatatcttttcagcggatctatacaaaccctactcctgctgacctctt
tcttccagtttctccgggtcttccttggattattattgcccatcttccgg
gttgtgcgtgtgtcagagacagctcgaacgataaatttctcaaaaccagt
actagagagggtgtgttgtgtgtgagaactgagtggagagttagcatgaa
ggctgcaaactagaaaggaaggtatgttctttcctttttgatccatcagg
ggagccccttctggtattaagatctttccggcacattgattttcatactt
tgtgatgaccctggaagaatcggcgtagcagcgtagcaccgctccatttt
ggtcttaccctcacctccccatgctatgaactgatcaatttcattgttct
tcatcacccttctcctagctttccacttccttcggatctcatgccatgtt
tctcagcatgaatcaaatttaattcgtgttttctacttccatatatactg
gaagaaatttaattagatctatttttgctcgggaggtcttcatactttga
gttctgatgccatcaccttatttcccccccccccttctcttgttctatct
tcttcctcatcttggcttgatcattttgatctgtcagttatagcatgatg
cattctcaatttgactgtatgtaagttcaaccggaaatatgttgaatgga
ttttctatatatcaacacttgatgtcaggcctgcatctgtttcgcttgtg
gtggtgtggccaaaattgtctatatttgatctttgctcttctttctcctc
atttcatgacgattcctactacggcttaaaccattctttattctttacta
atcatggatgttgcttgactcctagttgtttcgtactagctcaacttgga
gatcttttcattatttgcctagttggtgggtacgtttgtgacagatctaa
aatggtgcacgaaaagttttacttattatgaaaaaagggagcttaacagg
gtaatttctctatttattcgtgatgacattttttccttgataagggggat
tttttataatctgcactcacatgtttatatgtaaaatctagctcttttgt
tttgtttttggcatatttcccgctaagtatagagtttatgtggataacat
tataacttttcaagatccaatccacatctttgattgtgaaaatcatacaa
tagggaaaatcaactgaagggttaattagatgctatatgcatatatatat
atatgtgcgcgcgcgcgcgcctgaatttaactatgtatgcatccaactgt
ttcattgaaaaagatttgatatttttcagtctattctttttcgagtatat
atttaatatgtttcaatctgttttgaccattataagataaagcctatatt
caccaggcatttgagatgatcttttcatgcatgaaaaagctgttgttatc
acttcaactaaccagacgatctaacatgtatttgtataagaaacagacct
tgatttccttctgtaaaatcatgcatgtgttcgttttgaattggagtcgg
cgcgcctgtgttttgaccgtcaggaaagtcttttttttccctgaatagtc
aagggtctatacttcttgaagcaattgggacactaatcaattattgttta
tacctcggaccatcttttccttcttcacaccactaatcagtttatgcctt
ggaccattaattgtgttgttcacaagcttcttgtttatggtttacaaagc
attcgcctagatttgtgtgtgtctctacacatcgatcacttttaaatact
tgtcgctttcagttattcttttaacgtttggttatttatcttatttaaaa
aaattatcgtattattatttattttgtttgtgatttactttattatcaaa
agtatttcaaatatgacttatctttttttataagtgcactaatttttcaa
ataagatgaatggtcaaatgttacaagaaaaagttaaagcaaccactaat
ttagggcggaggtagtaaaacctagttattgtaaccaataattttatcaa
tctataaatgcaacacaaagtcacttcgtgatatctcacacaaagccact
tcaacgatgaaagctgactgcatgttttatcaaaacacatgtgatcagtt
tgttggatgaaaaaaattatctatgtcataaatcaagagttataatataa
gcttctggctctacaagtaacatttctatgttttttttttacgttcttac
atactatgttttgccaaaaaaaacatgatcattttgttggacgaaaagaa
atagtaaatatagagtgacctttgatatcattataatataagcttctgcc
tctataaataacatctatgcactttttacgtcgtagtaatttgatatatg
agaaatttacatataacatttttgtgcagcataaccacc

This promoter is a preferred promoter for use in the present invention.

2. Transcriptional Terminators

A variety of transcriptional terminators are available for use in expression cassettes. These are responsible for the termination of transcription beyond the transgene and correct mRNA polyadenylation. Appropriate transcriptional terminators are those that are known to function in plants and include the CAMV 35S terminator, the tml terminator, the nopaline synthase terminator and the pea rbcs E9 terminator. These can be used in both monocotyledons and dicotyledons. In addition, a gene's native transcription terminator may be used.

3. Sequences for the Enhancement or Regulation of Expression

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize Adhl gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes Develop. 1: 1183-1200 (1987)). In the same experimental system, the intron from the maize bronze 1 gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990)). Other leader sequences known in the art include but are not limited to: picomavirus leaders, for example, EMCV leader (Encephalomyocarditis 5′ noncoding region) (Elroy-Stein, O., Fuerst, T. R., and Moss, B. PNAS USA 86:6126-6130 (1989)); potyvirus leaders, for example, TEV leader (Tobacco Etch Virus) (Allison et al., 1986); MDMV leader (Maize Dwarf Mosaic Virus); Virology 154:9-20); human immunoglobulin heavy-chain binding protein (BiP) leader, (Macejak, D. G., and Samow, P., Nature 353: 90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie, D. R. et al., Molecular Biology of RNA, pages 237-256 (1989); and Maize Chlorotic Mottle Virus leader (MCMV) (Lommel, S. A. et al., Virology 81:382-385 (1991). See also, Della-Cioppa et al., Plant Physiology 84:965-968 (1987).

In addition to incorporating one or more of the aforementioned elements into the 5′ regulatory region of a target expression cassette of the invention, other elements peculiar to the target expression cassette may also be incorporated. Such elements include but are not limited to a minimal promoter. By minimal promoter it is intended that the basal promoter elements are inactive or nearly so without upstream activation. Such a promoter has low background activity in plants when there is no transactivator present or when enhancer or response element binding sites are absent. One minimal promoter that is particularly useful for target genes in plants is the Bz1 minimal promoter, which is obtained from the bronze 1 gene of maize. The Bz1 core promoter is obtained from the “myc” mutant Bz1-luciferase construct pBzlLucR98 via cleavage at the Nhel site located at −53 to −58. Roth et al., Plant Cell 3: 317 (1991). The derived Bz1 core promoter fragment thus extends from −53 to +227 and includes the Bz1 intron-1 in the 5′ untranslated region. Also useful for the invention is a minimal promoter created by use of a synthetic TATA element. The TATA element allows recognition of the promoter by RNA polymerase factors and confers a basal level of gene expression in the absence of activation (see generally, Mukumoto (1993) Plant Mol Biol 23: 995-1003; Green (2000) Trends Biochem Sci 25: 59-63)

4. Targeting of the Gene Product Within the Cell

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins which is cleaved during chloroplast import to yield the mature protein (e.g. Comai et al. J. Biol. Chem. 263: 15104-15109 (1988)). These signal sequences can be fused to heterologous gene products to effect the import of heterologous products into the chloroplast (van den Broeck, et al. Nature 313: 358-363 (1985)). DNA encoding for appropriate signal sequences can be isolated from the 5′ end of the cDNAs encoding the RUBISCO protein, the CAB protein, the EPSP synthase enzyme, the GS2 protein and many other proteins which are known to be chloroplast localized. See also, the section entitled “Expression With Chloroplast Targeting” in Example 37 of U.S. Pat. No. 5,639,949.

Other gene products are localized to other organelles such as the mitochondrion and the peroxisome (e.g. Unger et al. Plant Molec. Biol. 13: 411-418 (1989)). The cDNAs encoding these products can also be manipulated to effect the targeting of heterologous gene products to these organelles. Examples of such sequences are the nuclear-encoded ATPases and specific aspartate amino transferase isoforms for mitochondria. Targeting cellular protein bodies has been described by Rogers et al. (Proc. Natl. Acad. Sci. USA 82: 6512-6516 (1985)).

In addition, sequences have been characterized which cause the targeting of gene products to other cell compartments. Amino terminal sequences are responsible for targeting to the ER, the apoplast, and extracellular secretion from aleurone cells (Koehler & Ho, Plant Cell 2: 769-783 (1990)). Additionally, amino terminal sequences in conjunction with carboxy terminal sequences are responsible for vacuolar targeting of gene products (Shinshi et al. Plant Molec. Biol. 14: 357-368 (1990)).

By the fusion of the appropriate targeting sequences described above to transgene sequences of interest it is possible to direct the transgene product to any organelle or cell compartment. For chloroplast targeting, for example, the chloroplast signal sequence from the RUBISCO gene, the CAB gene, the EPSP synthase gene, or the GS2 gene is fused in frame to the amino terminal ATG of the transgene. The signal sequence selected should include the known cleavage site, and the fusion constructed should take into account any amino acids after the cleavage site which are required for cleavage. In some cases this requirement may be fulfilled by the addition of a small number of amino acids between the cleavage site and the transgene ATG or, alternatively, replacement of some amino acids within the transgene sequence. Fusions constructed for chloroplast import can be tested for efficacy of chloroplast uptake by in vitro translation of in vitro transcribed constructions followed by in vitro chloroplast uptake using techniques described by Bartlett et al. In: Edelmann et al. (Eds.) Methods in Chloroplast Molecular Biology, Elsevier pp 1081-1091 (1982) and Wasmann et al. Mol. Gen. Genet. 205: 446-453 (1986). These construction techniques are well known in the art and are equally applicable to mitochondria and peroxisomes.

The above-described mechanisms for cellular targeting can be utilized not only in conjunction with their cognate promoters, but also in conjunction with heterologous promoters so as to effect a specific cell-targeting goal under the transcriptional regulation of a promoter that has an expression pattern different to that of the promoter from which the targeting signal derives.

C. Construction of Plant Transformation Vectors

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptll gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

1. Vectors Suitable for Agrobacterium Transformation

Many vectors are available for transformation using Agrobacterium tumefaciens.

These typically carry at least one T-DNA border sequence and include vectors such as pBIN19 (Bevan, Nucl. Acids Res. (1984)). Below, the construction of two typical vectors suitable for Agrobacterium transformation is described.

a. pCIB200 and pCIB2001:

The binary vectors pCIB200 and pCIB2001 are used for the construction of recombinant vectors for use with Agrobacterium and are constructed in the following manner. pTJS75kan is created by Narl digestion of pTJS75 (Schmidhauser & Helinski, J. Bacteriol. 164: 446-455 (1985)) allowing excision of the tetracycline-resistance gene, followed by insertion of an Accl fragment from pUC4K carrying an NPTII (Messing & Vierra, Gene 19: 259-268 (1982): Bevan et al., Nature 304: 184-187 (1983): McBride et al., Plant Molecular Biology 14: 266-276 (1990)). XhoI linkers are ligated to the EcoRV fragment of PCIB7 which contains the left and right T-DNA borders, a plant selectable nos/nptll chimeric gene and the pUC polylinker (Rothstein et al., Gene 53: 153-161 (1987)), and the XhoI-digested fragment are cloned into SalI-digested pTJS75kan to create pCIB200 (see also EP 0 332 104, example 19). pCIB200 contains the following unique polylinker restriction sites: EcoRI, SstI, KpnI, BgmlI, Xbal, and SalI. pCIB2001 is a derivative of pCIB200 created by the insertion into the polylinker of additional restriction sites. Unique restriction sites in the polylinker of pCIB2001 are EcoRI, SstI, KpnI, BglII, Xbal, SalI, MluI, BclI, AvrII, ApaI, HpaI, and Stul. pCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

b. pCIB10 and Hygromycin Selection Derivatives Thereof:

The binary vector pCIB 10 contains a gene encoding kanamycin resistance for selection in plants and T-DNA right and left border sequences and incorporates sequences from the wide host-range plasmid pRK252 allowing it to replicate in both E. coli and Agrobacterium. Its construction is described by Rothstein et at (Gene 53: 153-161 (1987)). Various derivatives of pCIB10 are constructed which incorporate the gene for hygromycin B phosphotransferase described by Gritz et al. (Gene 25: 179-188 (1983)). These derivatives enable selection of transgenic plant cells on hygromycin only (pCIB743), or hygromycin and kanamycin (pCIB715, pCIB717).

2. Vectors Suitable for Non-Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Below, the construction of typical vectors suitable for non-Agrobacterium transformation is described.

a. pCIB3064:

pCIB3064 is a pUC-derived vector suitable for direct gene transfer techniques in combination with selection by the herbicide basta (or phosphinothricin). The plasmid pCIB246 comprises the CaMV 35S promoter in operational fuson to the E. coli GUS gene and the CaMV 35S transcriptional terminator and is described in the PCT published application WO 93/07278. The 35S promoter of this vector contains two ATG sequences 5′ of the start site. These sites are mutated using standard PCR techniques in such a way as to remove the ATGs and generate the restriction sites SspI and PvuII. The new restriction sites are 96 and 37 bp away from the unique SalI site and 101 and 42 bp away from the actual start site. The resultant derivative of pCIB246 is designated pCIB3025. The GUS gene is then excised from pCIB3025 by digestion with SalI and SacI, the termini rendered blunt and religated to generate plasmid pCIB3060. The plasmid pJIT82 is obtained from the John Innes Centre, Norwich and the a 400 bp SmaI fragment containing the bar gene from Streptomyces vifidochromogenes is excised and inserted into the HpaI site of pCIB3060 (Thompson et al. EMBO J. 6: 2519-2523 (1987)). This generated pCIB3064, which comprises the bar gene under the control of the CaMV 35S promoter and terminator for herbicide selection, a gene for ampicillin resistance (for selection in E. coli) and a polylinker with the unique sites SphI, PstI, HindIII, and BamHI. This vector is suitable for the cloning of plant expression cassettes containing their own regulatory signals.

b. pSOG19 and pSOG35:

pSOG35 is a transformation vector that utilizes the E. coli gene dihydrofolate reductase (DFR) as a selectable marker conferring resistance to methotrexate. PCR is used to amplify the 35S promoter (−800 bp), intron 6 from the maize Adh1 gene (−550 bp) and 18 bp of the GUS untranslated leader sequence from pSOG10. A 250-bp fragment encoding the E. coli dihydrofolate reductase type II gene is also amplified by PCR and these two PCR fragments are assembled with a SacI-PstI fragment from pB 1221 (Clontech) which comprises the pUC 19 vector backbone and the nopaline synthase terminator. Assembly of these fragments generates pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus (MCMV) generates the vector pSOG35. pSOG19 and pSOG35 carry the pUC gene for ampicillin resistance and have HindIII, SphI, PstI and EcoRI sites available for the cloning of foreign substances.

3. Vector Suitable for Chloroplast Transformation

For expression of a nucleotide sequence of the present invention in plant plastids, plastid transformation vector pPH143 (WO 97/32011, example 36) is used. The nucleotide sequence is inserted into pPH143 thereby replacing the PROTOX coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

D. Transformation

Once a nucleic acid sequence of the invention has been cloned into an expression system, it is transformed into a plant cell. The receptor and target expression cassettes of the present invention can be introduced into the plant cell in a number of art-recognized ways. Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

1. Transformation of Dicotyledons

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend of the complement of vir genes carried by the host Agrobacterium strain either on a co-resident Ti plasmid or chromosomally (e.g. strain CIB542 for pCIB200 and pCIB2001 (Uknes et al. Plant Cell 5: 159-169 (1993)). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen & Willmitzer, Nucl. Acids Res. 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792 all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

2. Transformation of Monocotyledons

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e. co-transformation) and both these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4: 1093-1096 (1986)).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) have published techniques for transformation of A188-derived maize line using particle bombardment. Furthermore, WO 93/07278 and Koziel et al. (Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. Plant Cell Rep 7: 379-384 (1988); Shimamoto et al. Nature 338: 274-277 (1989); Datta et al. Biotechnology 8: 736-740 (1990)). Both types are also routinely transformable using particle bombardment (Christou et al. Biotechnology 9: 957-962 (1991)). Furthermore, WO 93/21335 describes techniques for the transformation of rice via electroporation.

Patent Application EP 0 332 581 describes techniques for the generation, transformation and regeneration of Pooideae protoplasts. These techniques allow the transformation of Dactylis and wheat. Furthermore, wheat transformation has been described by Vasil et al. (Biotechnology 10: 667-674 (1992)) using particle bombardment into cells of type C long-term regenerable callus, and also by Vasil et al. (Biotechnologyl 11:

1553-1558 (1993)) and Weeks et al. (Plant Physiol. 102: 1077-1084 (1993)) using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, Physiologia Plantarum 15: 473-497 (1962)) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 hours and are then bombarded. Twenty embryos per target plate is typical, although not critical. An appropriate gene-carrying plasmid (such as pCIB3064 or pSG35) is precipitated onto micrometer size gold particles using standard procedures. Each plate of embryos is shot with the DuPont Biolistics.®.) helium device using a burst pressure of about. 1000 psi using a standard 80 mesh screen. After bombardment, the embryos are placed back into the dark to recover for about 24 hours (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Tranformation of monocotyledons using Agrobacterium has also been described. See, WO 94/00977 and U.S. Pat. No. 5,591,616, both of which are incorporated herein by reference. See also, Negrotto et al., Plant Cell Reports 19: 798-803 (2000), incorporated herein by reference.

For example, rice (Oryza sativa) can be used for generating transgenic plants. Various rice cultivars can be used (Hiei et al., 1994, Plant Journal 6:271-282; Dong et al., 1996, Molecular Breeding 2:267-276; Hiei et al., 1997, Plant Molecular Biology, 35:205-218). Also, the various media constituents described below may be either varied in quantity or substituted. Embryogenic responses are initiated and/or cultures are established from mature embryos by culturing on MS-CIM medium (MS basal salts, 4.3 g/liter; B5 vitamins (200.times.), 5 ml/liter; Sucrose, 30 g/liter; proline, 500 mg/liter; glutamine, 500 mg/liter; casein hydrolysate, 300 mg/liter; 2,4-D (1 mg/ml), 2 ml/liter; adjust pH to 5.8 with 1 N KOH; Phytagel, 3 g/liter). Either mature embryos at the initial stages of culture response or established culture lines are inoculated and co-cultivated with the Agrobacterium tumefaciens strain LBA4404 (Agrobacterium) containing the desired vector construction. Agrobacterium is cultured from glycerol stocks on solid YPC medium (100 mg/L spectinomycin and any other appropriate antibiotic) for .about.2 days at 28.degree. C. Agrobacterium is re-suspended in liquid MS-CIM medium. The Agrobacterium culture is diluted to an OD600 of 0.2-0.3 and acetosyringone is added to a final concentration of 200 uM. Acetosyringone is added before mixing the solution with the rice cultures to induce Agrobacterium for DNA transfer to the plant cells. For inoculation, the plant cultures are immersed in the bacterial suspension. The liquid bacterial suspension is removed and the inoculated cultures are placed on co-cultivation medium and incubated at 22.degree. C. for two days. The cultures are then transferred to MS-CIM medium with Ticarcillin (400 mg/liter) to inhibit the growth of Agrobacterium. For constructs utilizing the PMI selectable marker gene (Reed et al., In Vitro Cell. Dev. Biol.-Plant 37:127-132), cultures are transferred to selection medium containing Mannose as a carbohydrate source (MS with 2% Mannose, 300 mg/liter Ticarcillin) after 7 days, and cultured for 3-4 weeks in the dark. Resistant colonies are then transferred to regeneration induction medium (MS with no 2,4-D, 0.5 mg/liter IAA, 1 mg/liter zeatin, 200 mg/liter timentin 2% Mannose and 3% Sorbitol) and grown in the dark for 14 days. Proliferating colonies are then transferred to another round of regeneration induction media and moved to the light growth room. Regenerated shoots are transferred to GA7 containers with GA7-1 medium (MS with no hormones and 2% Sorbitol) for 2 weeks and then moved to the greenhouse when they are large enough and have adequate roots. Plants are transplanted to soil in the greenhouse (To generation) grown to maturity, and the T.sub. 1 seed is harvested.

3. Transformation of Plastids

Seeds of Nicotiana tabacum c.v. ‘Xanthienc’ are germinated seven per plate in a 1″ circular array on T agar medium and bombarded 12-14 days after sowing with 1 .mu.m tungsten particles (M10, Biorad, Hercules, Calif.) coated with DNA from plasmids pPH143 and pPH145 essentially as described (Svab, Z. and Maliga, P. (1993) PNAS 90, 913-917). Bombarded seedlings are incubated on T medium for two days after which leaves are excised and placed abaxial side up in bright light (350-500 .mu.mol photons/m.sup.2/s) on plates of RMOP medium (Svab, Z., Hajdukiewicz, P. and Maliga, P. (1990) PNAS 87, 8526-8530) containing 500 mu.g/ml spectinomycin dihydrochloride (Sigma, St. Louis, Mo.). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). BamHI/EcoRI-digested total cellular DNA (Mettler, I. J. (1987) Plant Mol Biol Reporter 5, 346349) is separated on 1% Tris-borate (TBE) agarose gels, transferred to nylon membranes (Amersham) and probed with .sup.32P-labeled random primed DNA sequences corresponding to a 0.7 kb BamHI/HindIII DNA fragment from pC8 containing a portion of the rps 7/12plastid targeting sequence. Homoplasmic shoots are rooted aseptically on spectinomycin-containing MS/IBA medium (McBride, K. E. et al. (1994) PNAS 91, 7301-7305) and transferred to the greenhouse.

V. Breeding and Seed Production

A. Breeding

The plants obtained via transformation with a nucleic acid sequence of the present invention can be any of a wide variety of plant species, including those of monocots and dicots; however, the plants used in the method of the invention are preferably selected from the list of agronomically important target crops set forth supra. The expression of a gene of the present invention in combination with other characteristics important for production and quality can be incorporated into plant lines through breeding. Breeding approaches and techniques are known in the art. See, for example, Welsh J. R., Fundamentals of Plant Genetics and Breeding, John Wiley & Sons, NY (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D. P., Breeding for Resistance to Diseases and Insect Pests, Springer-Verlag, NY (1986); and Wricke and Weber, Quantitative Genetics and Selection Plant Breeding, Walter de Gruyter and Co., Berlin (1986).

The genetic properties engineered into the transgenic seeds and plants described above are passed on by sexual reproduction or vegetative growth and can thus be maintained and propagated in progeny plants. Generally said maintenance and propagation make use of known agricultural methods developed to fit specific purposes such as tilling, sowing or harvesting, Specialized processes such as hydroponics or greenhouse technologies can also be applied. As the growing crop is vulnerable to attack and damages caused by insects or infections as well as to competition by weed plants, measures are undertaken to control weeds, plant diseases, insects, nematodes, and other adverse conditions to improve yield. These include mechanical measures such a tillage of the soil or removal of weeds and infected plants, as well as the application of agrochemicals such as herbicides, fungicides, gametocides, nematicides, growth regulants, ripening agents and insecticides.

Use of the advantageous genetic properties of the transgenic plants and seeds according to the invention can further be made in plant breeding, which aims at the development of plants with improved properties such as tolerance of pests, herbicides, or stress, improved nutritional value, increased yield, or improved structure causing less loss from lodging or shattering. The various breeding steps are characterized by well-defined human intervention such as selecting the lines to be crossed, directing pollination of the parental lines, or selecting appropriate progeny plants. Depending on the desired properties, different breeding measures are taken. The relevant techniques are well known in the art and include but are not limited to hybridization, inbreeding, backcross breeding, multi-line breeding, variety blend, interspecific hybridization, aneuploid techniques, etc. Hybridization techniques also include the sterilization of plants to yield male or female sterile plants by mechanical, chemical, or biochemical means. Cross pollination of a male sterile plant with pollen of a different line assures that the genome of the male sterile but female fertile plant will uniformly obtain properties of both parental lines. Thus, the transgenic seeds and plants according to the invention can be used for the breeding of improved plant lines, that for example, increase the effectiveness of conventional methods such as herbicide or pesticide treatment or allow one to dispense with said methods due to their modified genetic properties. Alternatively new crops with improved stress tolerance can be obtained, which, due to their optimized genetic “equipment”, yield harvested product of better quality than products that were not able to tolerate comparable adverse developmental conditions.

B. Seed Production

In seed production, germination quality and uniformity of seeds are essential product characteristics. As it is difficult to keep a crop free from other crop and weed seeds, to control seed-borne diseases, and to produce seed with good germination, fairly extensive and well-defined seed production practices have been developed by seed producers, who are experienced in the art of growing, conditioning and marketing of pure seed. Thus, it is common practice for the farmer to buy certified seed meeting specific quality standards instead of using seed harvested from his own crop. Propagation material to be used as seeds is customarily treated with a protectant coating comprising herbicides, insecticides, fungicides, bactericides, nematicides, molluscicides, or mixtures thereof. Customarily used protectant coatings comprise compounds such as captan, carboxin, thiram (TMTD.®.), methalaxyl (Apron.®.), and pirimiphos-methyl (Actellic.®.). If desired, these compounds are formulated together with further carriers, surfactants or application-promoting adjuvants customarily employed in the art of formulation to provide protection against damage caused by bacterial, fungal or animal pests. The protectant coatings may be applied by impregnating propagation material with a liquid formulation or by coating with a combined wet or dry formulation. Other methods of application are also possible such as treatment directed at the buds or the fruit.

The following examples are given by way of illustration and explanation, and are not intended to be limiting in any way.

### EXAMPLES

### Example 1

Agrobacterium Transformation of Maize—Immature embryos

Preparation of Ear

Harvest ears when immature embryos in the center kernels are approximately 0.5-1.0 mm.

Shuck and sterilize ears in a solution of 20% Chlorox and 3 drops Tween/liter of solution. Put on an orbital shaker for 20 minutes.

Rinse ears three times with sterile ddH₂O.

In a sterile environment cut off the tops of the kernels. Rest the ear on a sterile Petri dish and isolate the immature embryos.

Preparing Inoculation Solution for Transformation.

To 100 mL of LP-Lsinf. Medium, add 50 μl of acetosyringone (AS) stock solution (40 mg/ml stock/mL) for a final concentration of 100 μM AS.
Pipet 4 ml (2.5) of the infection medium into a 10 ml disposable tube.
Set up Eppendorf tubes for collecting the embryos at this time and add ˜1.4 ml infection medium with AS to them.
   Preparation of Agrobacterium Suspension
Take one loop of Agrobacterium and re-suspend it by vortex in 10 ml disposable tube with 4 ml (2.5 ml) infection medium.
Measure optical density of the Agrobacterium suspension. Adjust the OD₆₆₀ to approximately 0.45 to 0.55.
   Isolation of Immature Embryos and Transformation
Excise embryos and place them on top of the infection medium in an eppendorf tube.
Excise embryos for 30-45 minutes to obtain a total of ˜150 embryos.
Vortex embryos (or hand shake) for 5 seconds.
Heat shock the embryos in a 45° C. water bath for 5 minutes. Do not have lid of eppendorf tube in contact with water (possible contamination issues).
Using a disposable pipet remove infection medium and replace with 1.5 mL Agrobacterium suspension. Vortex for 30 seconds.
Allow the tube to sit for 5 minutes.
Shake the tube to suspend embryos and pour into a Petri dish with LS modified As 500 medium.
Pipet off Agrobacterium suspension and transfer embryos to an area of the plate that has not been exposed to Agrobacterium.

Make absolutely sure that the embryos are all scutellum side up.

Co-cultivation

Co-culture embryos and Agrobacterium at 23° C. for 2-3 days.
   Callus/somatic Embryo Induction
Transfer tissue (18 embryos/plate) to pre-selection/callus induction medium for 10 to 14 days at 28° C. in the dark.
   Mannose Selection
Transfer callus clusters on Selection medium. 9 clusters per plate. Culture for approximately 2 weeks at 28° C. in the dark.
Check cultures for contamination and callus response and culture for additional 2 weeks at 28° C. in the dark.

Transfer 4 events per plate of growing tissue to MS Regeneration (R1) medium and leave in the dark for 10-14 days.

Transfer growing tissue/plants of 4 events per plate to light for 14 days in light.

Transfer events to rooting media in tissue culture containers (2 events/Greiner containers).

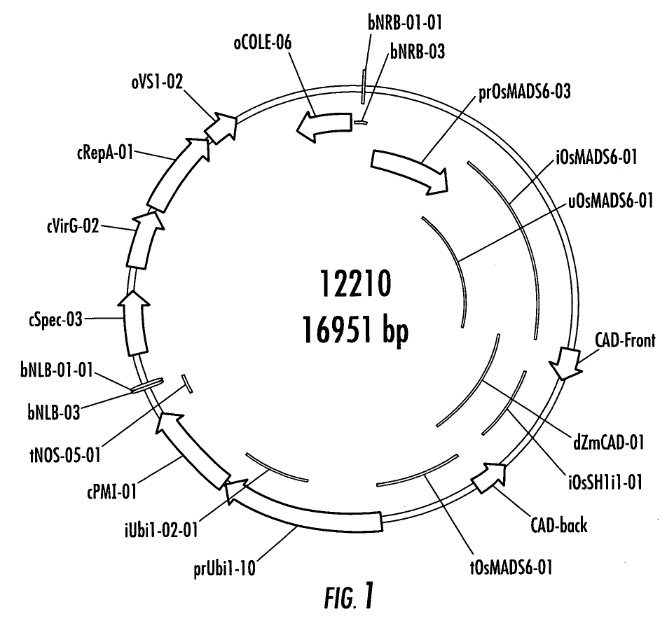

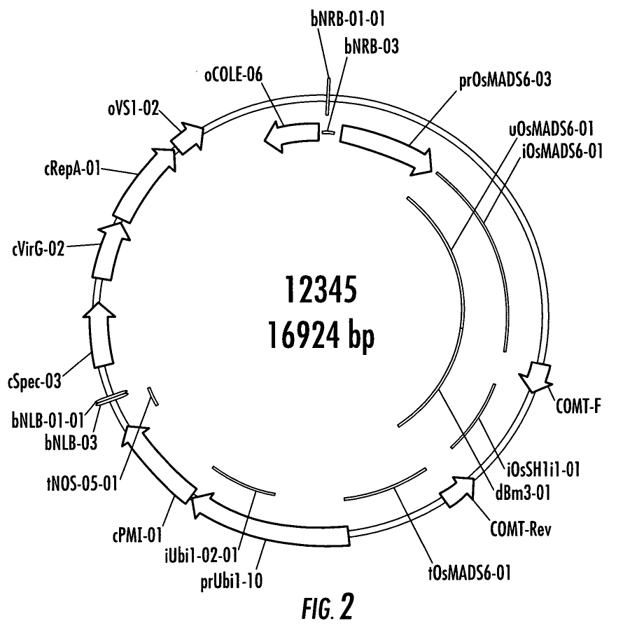

Transgenic maize was grown in the greenhouse to the T0 or T1 stage, and cob samples and other materials were selected from the transgenic events produced. The plasmids shown in FIG. 1 and FIG. 2 show the plasmids (pSyn 12210 containing the CAD RNAi construct, and pSyn12345, containing the COMT RNAi construct) used in the maize transformation protocol given above.

### Example 2

T1 CAD event selection pSyn12210

T1 cob samples of a total 15 events (10 low, 3 medium and 2 high copy) include 65 lines (28 low, 14 medium, 5 high copy and 18 null control lines) were sent to MSU for NDF (fiber), ADL (lignin), IVNDFD (in vitro NDF digestibility) analysis. 3 BM3 isolines and 2 hybrid checks were also included. We compared the difference among the lines (transgenic vs. null) of the same event, among the events, or among low, medium and high copy events with the BM3 positive or negative controls and hybrid check. Although a few of these made it into the top 16 lines mentioned below, none of them showed consistent reduction in lignin in future tests. The data are in Table 1, and the methods used for analysis are given below.

T1 COMT Event Selection (pSyn 12345)

T1 cob analysis: total 41 events (23 low, 13 medium, and 5 high copy) include 131 lines (24 low, 13 medium, 8 high copy and 20 null control lines) were sent to MSU for analysis (ADL, NDF, IVNDFD). 3BM3 isolines, one JHAX707 control, and one hybrid check were also included.

T1 Cob analysis: The top 7 lines containing pSyn12210 or pSyn12345 were selected based on the lignin content and in vitro digestibility data. These lines showed a reduction in the lignin content and improved digestibility compared to the control cob, as shown in Table 1. Data pertaining to he two best events containing plasmid pSyn12345 are presented in Table 2.

TABLE 1
Silage characteristics of select events containing RNAi knockouts of either CAD or COMT
In 2 genetic backgrounds as compared to null lines from the same event.

| | Gene | | | DM % | ASH % | NDF % | ADF % | Lignin % | IVTD % | IVNDFD % |
|---|---|---|---|---|---|---|---|---|---|---|
| Event | Knockout | Inbred | Generation | MEAN | MEAN | MEAN | MEAN | MEAN | MEAN | MEAN |
| 1 | Null | 1 | T2 | 89.4 | 2.6 | 67.0 | 37.1 | 5.0 | 59.6 | 39.9 |
| | CAD | | | 89.3 | 3.1 | 64.9 | 35.2 | 4.3 | 64.3 | 45.0 |
| | | | | −0.1 | 0.5 | −2.1 | −1.9 | −0.7 | 4.7 | 5.1 |
| 2 | Null | 1 | T2 | 90.5 | 1.2 | 76.6 | 41.9 | 5.5 | 53.0 | 38.7 |
| | CAD | | | 89.8 | 2.7 | 70.9 | 39.3 | 5.1 | 59.8 | 43.5 |
| | | | | −0.7 | 1.5 | −5.7 | −2.6 | −0.5 | 6.7 | 4.8 |
| 3 | Null | 1 | T2 | 94.0 | 3.2 | 77.1 | 44.7 | 6.6 | 49.4 | 34.4 |
| | COMT | | | 94.3 | 3.1 | 75.6 | 43.7 | 5.8 | 53.5 | 38.5 |
| | | | | 0.3 | −0.1 | −1.5 | −0.9 | −0.8 | 4.1 | 4.1 |
| 4 | Null | 1 | T1 | 92.4 | 2.5 | 77.1 | 45.2 | 6.8 | 48.8 | 33.6 |
| | COMT | | | 90.1 | 3.4 | 75.6 | 43.2 | 6.0 | 53.3 | 38.3 |
| | | | | −2.2 | 0.9 | −1.5 | −2.1 | −0.8 | 4.5 | 4.7 |
| 4 | Null | 1 | T2 | 88.8 | 3.4 | 78.9 | 45.3 | 6.6 | 50.1 | 36.7 |
| | COMT | | | 87.7 | 3.1 | 72.7 | 40.6 | 5.7 | 57.0 | 40.9 |
| | | | | −1.1 | −0.2 | −6.2 | −4.7 | −0.9 | 7.0 | 4.2 |
| 5 | Null | 1 | T2 | 89.0 | 2.7 | 78.2 | 44.8 | 6.8 | 51.0 | 37.4 |
| | COMT | | | 88.8 | 3.4 | 70.5 | 39.2 | 5.6 | 59.0 | 42.2 |
| | | | | −0.2 | 0.7 | −7.7 | −5.6 | −1.2 | 7.9 | 4.7 |
| 6 | Null | 1 | T2 | 89.8 | 2.4 | 82.6 | 46.7 | 6.6 | 43.3 | 31.4 |
| | COMT | | | 89.6 | 3.1 | 78.8 | 43.7 | 5.8 | 50.8 | 37.6 |
| | | | | −0.2 | 0.6 | −3.9 | −3.0 | −0.8 | 7.5 | 6.2 |
| 7 | Null | 2 | T1 | n/a | n/a | n/a | n/a | 6.3 | n/a | n/a |
| | COMT | | | n/a | n/a | n/a | n/a | 5.6 | n/a | n/a |
| | | | | | | | | −0.7 | | |
| | Bmr | | | 89.5 | 3.4 | 84.7 | 48.3 | 6.1 | 47.7 | 38.3 |
| | isogenic | | | | | | | | | |
| | normal | | | | | | | | | |
| | Bmr | | | 89.5 | 3.2 | 80.1 | 42.4 | 1.7 | 73.2 | 66.6 |
| | | | | 0 | −0.2 | −4.6 | −5.9 | −4.4 | 25.5 | 28.3 |

DM = dry matter,
NDF = Neutral Detergent Fiber,
ADF = Acid Detergent Fiber,
IVTD = In vitro true digestibility,
IVNDFD = In vitro NDF disappearance.

TABLE 2
Data showing a consistent reduction in % lignin and increase in
% IVNDFD for top 2 events

| | | Lignin (%) | | IVNDFD (%) | | |
|---|---|---|---|---|---|---|
| | | Expt 1 | Expt 2 | Expt 1 | Expt 2 | Expt 3 |
| Event | | T1 | T2 | T1 | T2 | T3 |
| 3 | Null | 6.6 | 5.6 | 34.4 | 35.8 | 27.1 |
| | COMT | 5.8 | 4.9 | 38.5 | 40.0 | 31.4 |
| 6 | Null | 6.6 | — | 31.4 | 33.0 | — |
| | COMT | 5.8 | 6.2 | 37.6 | 40.3 | 31.6 |
| bm3* | | 1.7 | 1.2 | 66.6 | 68.0 | 53.5 |
| bm3 iso | | 6.1 | 4.6 | 38.3 | 41.2 | 34.2 |

For general methodologies applicable to these analyses see:

Goering, H. K., and P. J. Van Soest. 1970. Forage Fiber Analyses. Apparatus, Reagents, Procedures, and some applications. Agric. Handbook No. 379. ARS-USDA.

ADF—Acid Detergent Fiber—(Van Soest Method)

Performed under chemical hood, with proper equipment that includes chemical resistant apron, lab coat, chemical resistant rubber gloves, safety glasses.

The ADF method hydrolyzes components of the cell wall that includes pectins and hemicelluloses. The remaining fraction, called ADF contains cellulose, lignin and ash. It is expressed as a percentage of the total cell wall fraction.

The ADL residue is generated from the ADF fraction, and it represents lignin+ash.

The initial step is to recover the cell wall fraction from a particular plant tissue. In other words it will eliminate soluble compounds such as sugars, proteins, oil, and soluble fiber. If the sample contains a large amount of oil (such as soybean seeds) then a wash with acetone is required (grind first, then use a glass tube, or short incubation time in a plastic tube). The tissue is dried and ground in particles of no more than 2 mm in size. Typically about 2-3 gr of tissue is added to a 50 mL conical tube. Then 40 mL of 80% ethanol is added, and mixed for 5 hrs on a rotary shaker. Another wash is required overnight. Then 40 mL of water is added for a 1 hr wash, repeated for a total of 3 washes. Some tissue will be lost during changes of solution. A preliminary wash should be done to estimate the amount of initial tissue to produce about 1 gr of final cell wall sample.

In the case of cob tissue, because the cell wall content is very high, it is not required to do a cell wall preparation. So the ADF and ADL analyses are calculated on a total dry matter basis.

ADF Solution

2% CTAB into 1 N H₂SO4

(for 1 L stock, add 20 gr CTAB to 900 mL deionized (di) water, stir for few min until particle size reduced. Then add 62.5 mL 16 N sulfuric acid (Fisher A298-212, 98% density 1.84 g/mL). As you add the acid, the mixture CTAB+water/acid becomes solubilized. Fill up to 1 L with di water.

The ADF solution can also be purchased at ANKOM (www.ankom.com).

Record weight of ANKOM bag

Weigh 250 mg of air dried sample (from o/n incubation at 37C of dry sample)

Pour in ANKOM filter bag (F57 type); record weight

Seal with heat sealer

Add to a 2000 mL Pyrex glass beaker, typically 20 bags (24 max) with 500 mL ADF solution. Cover with 1000 mL Pyrex dish. Place into glass dish (Pyrex 190 mm diameter×100 mm height), filled with boiling water. Also add some PTFE boiling stones in dish. Water should keep boiling for one hour. Agitate bags every 15 minutes.

Rinse minimum 4 times with 85-90C H₂O. All liquid waste is disposed in sink with continuous water flow. Another 2 minutes rinse with small volume of acetone, which is then disposed of in solvent waste container.

Dry in 70C oven overnight.

Weight residual tissue=ADF

ADL—Acid Detergent Lignin

Performed under chemical hood, with proper equipment that includes chemical resistant apron, lab coat, chemical resistant rubber gloves, safety glasses.

To the dried ADF residue (20-24 bags), add 500 mL of 72% sulfuric acid, to cover all bags.

Use a 2000 mL Pyrex glass beaker, with 20 bags (24 max) and over with 1000 mL Pyrex dish

NOTE: 72% sulfuric acid can be prepared by adding 750 mL of concentrated (95% acid) into 250 mL water. Caution: the mix is verv hot and requires pouring the acid very slowly to avoid projections. Also the bottle is then put into a room temperature water bath (2 rinses) for rapid cooling and use. You can also buy the 72% H2SO4 acid from Ankom.

Incubate at RT for 3 hours, stir several times.

Excess acid is disposed of in liquid acid waste.

Then rinse at least 5 times with hot water (85 to 90C). Washes are performed in the sink.

Final rinse in acetone for 2 minutes, disposed of solvent in waste container.

Dry in oven at 70C overnight.

Weight residual tissue=ADL

Neutral Detergent Fiber Analysis

Prepare the samples as the standard grinding protocol recommends, without using the Perten Hammer Mill.

Place the samples in the dry balance to assess moisture content.

Number bags with solvent resistant marker.

Record weight of empty Ankom dry sample bags.

Take dried sample and weigh 0.5 g of dried sample into Ankom filter bags.

Seal the bags with the heat sealer.

Dissolve 20 g of Sodium Sulfite (0.5 g/50 ml of NDF solution) into 2000 ml of NDF solution.

Turn on heat until boil is achieved, once boiling add sample in bags and cover loosely.

Set timer for 105 minutes.

After time is up pour samples into strainer over NDF waste container.

Rinse slowly with 2L's of 85*-90*C water.

Repeat the rinse process for a total of three rinses.

Add cold water to samples to aid in cooling.

Drain bags and place them in acetone for three minutes.

Spread out bags and allow drying, in the oven is fine after most of the acetone is gone.

Weigh bags, collect data in designated NDF Spreadsheet.

In Vitro True Digestibility Determination

1. Calibrate the balance and weigh 0.5 g (1.0 g for rates of digestion) dry ground (1 mm screen) sample into 125 ml Erlenmeyer flasks. Prepare 6 standard samples for each bath used.

2. Prepare media by adding ingredients below in order:

Number of flasks: 24 48 78 110 166

Distilled water 500 ml 1.00 l 1.75 l 2.25 l 3.50 l

Trypticase® Peptonea 2.5 g 5.0 g 8.75 g 11.25 g 17.5 g

Micromineral solution 0.125 ml 0.25 ml 0.438 ml 0.563 ml 0.880 ml

Rumen buffer solution 250 ml 500 ml 875 ml 1.125 l 1.75 l

Macromineral solution 250 ml 500 ml 875 ml 1.125 l 1.75 l

Resazurin 1.25 ml 2.5 ml 4.38 ml 5.63 ml 8.80 ml

1.00 l 2.00 l 3.50 l 4.50 l 7.00 l

Mix and add 40 ml per flask. The media should be added to flasks at least 1 h before inoculation to hydrate the samples. Heat water baths overnight.

3. Prepare reducing solution:

Add cysteine HCl, H₂O, and NaOH and dissolve. Add Na2S.9H₂O and dissolve again.

4. While reducing solution is mixing, arrange flasks in water bath. Place flasks with one standard sample in each row arranged diagonally across the water bath. Add 2 ml of reducing solution to each flask with Eppendorf repeater pipette. Stopper each flask with a CO₂ flushing tube. Turn on CO₂ and allow samples to reduce (red color turns clear or tea colored), before addition of inocula.

5. Prepare Inoculum:

Collect rumen fluid and ingesta from two fistulated animals 2 hours after feeding (cows are fed 7:00 am, collect at 9:15 am). Keep fluid in a clean thermal container which has been preheated by hot tap water. Pour water out into a bucket and place the cannula plug into it to keep it pliable. Form a tunnel through the rumen mat to allow a plastic cup to reach to the ventral rumen. Place a layer of ingesta over the fluid and cover with a lid to eliminate airspace. Replace cannula plugs tightly. Transport fluid to lab and place under CO2. Approximately 2 l unprocessed fluid is needed for a set of 166 samples. Blend fluid and ingesta in the 1 gallon Waring blender taking care to flush with CO2 continuously. Line a large plastic Buchner funnel with 1 layer of nylon mesh and pass the blended inocula through it. Squeeze well.

a Use only Trypticase™ Peptone pancreatic digest of casein (Becton Dickinson BBL #4311921).

Number of flasks: 24 48 78 110 166

Distilled water 48 ml 95 ml 167 ml 261 ml 356 ml

L(+)Cysteine HCl.H2O 313 mg 625 mg 1.094 g 1.719 g 2.344 g 1 N NaOH 2 ml 4 ml 7 ml 11 ml 15 ml

Na2S.9H2O 313 mg 625 mg 1.094 g 1.719 g 2.344 g 50 ml 100 ml 175 ml 275 ml 375 ml

Pass inocula through glass wool into a large plastic beaker to filter small particles. The filtrate must also be kept under CO2 at all times. Transfer inocula to a bottle that the 50 ml Brinkman pipetter attaches to.

Using a Brinkman pipetter, inoculate each flask by first removing the bunsen valve, injecting 10 ml of fluid, and replacing the valve. This procedure will flush each flask with CO2 and displace any O2 that may be present. Swirl the bottle containing rumen fluid frequently during inoculation to keep particles suspended.

6. Seal flasks, notice and correct any CO2 leaks, and adjust CO2 pressure to just enough to produce slow bubbles in the manometer. Water bath temperature throughout the fermentation should be kept at 40° C. (100-102° F.). Digest samples for 30 hr unless otherwise specified. To stop fermentation, remove flasks from bath and add 20 ml of ND solution with the Unispense automatic dispenser. Put a cork on each flask to prevent spilling and store samples in refrigerator or immediately do NDF procedure on the samples if rates are being calculated.

7. Wash flask contents with 80 ml neutral-detergent into a 600 ml Berzelius beaker. Add 0.5 g sodium sulfite. Reflux for 1 hour, timed from the onset of boiling. Add approximately 1 teaspoon of acid purified sea sand (Seesand, Fluka Chemika #84880) into clean Gooch crucibles. Filter sample through a clean, numbered Gooch crucible as in NDF procedure. Wash and rinse with hot water until foam disappears and twice with acetone.

Allow acetone to completely evaporate, dry crucibles overnight at 100° C., calibrate the balance and hot weigh. Ash samples at 500° C. for 6 hr, cool to 200° C., transfer to drying oven and hot weigh crucible plus ash.

8. Calculate in vitro true digestibility of dry matter:
IVTD=[1−(N−CA)/S]×100 where N=crucible+ND residue weight
CA=empty crucible weight
S=sample dry matter weight

9. Calculate in vitro NDF digestibility:
IVCWD=[1−((N−CA)/(S×F/100))]×100 where N=crucible+ND residue weight
CA=empty crucible weight
S=sample dry matter weight
F=percent NDF of sample

10. To calculate rates of digestion, prepare a set of 13 samples to be incubated from 0 to 120 hours. Place all flasks in water baths and remove with time. Process residues as listed above. Calculate NDF remaining as

a percentage of original NDF for each sample. Either use a non-linear regression method (JMP or SAS) or a log-transform procedure as follows: Subtract the indigestible (˜120 hour) residue from each fraction.

Calculate the natural log (ln) of each point and calculate linear regression of the plot.

Slope of this line is the rate of digestion of the fraction in question.

This method is a modification of the Tilley-Terry in vitro apparent digestibility procedure. Steps 1 through 5 are common to both techniques. From step 5, continue below with step 6a for the Tilley-Terry method.

6a. After a 48 hour fermentation, carefully add 2 ml 6N HCl to each flask to avoid excessive foaming. This will lower the pH to below 2. Add 0.5 g pepsin, and swirl to dissolve. Add 1 ml toluene, replace flasks in water bath, and incubate another 48 hours.

7a. Remove flasks from water bath and filter on previously tared Whatman #4, 41 or 54 paper without applying vacuum. Rinse filter paper twice by filling with hot water and allowing to drain. Fill filter with acetone, allow to drain and air-dry. Fold papers, dry at 100° C., and weigh. Use a dry matter factor, calculated on separate papers, to correct for tare on papers used for filtering. Separate blanks, containing inocula and medium but no sample, and standard forage samples should also be analyzed.

8a. Calculate in vitro apparent digestibility (Tilley-Terry):
IVDMD=[1−(R−F)−B]×100 where R=weight of filter paper and residue
F=weight of filter paper
B=blank sample weight

### REFERENCES

Goering, H. K. and P. J. Van Soest. 1970. Forage and Fiber Analysis. Agricultural Handbook no. 379. U.S. Dept. Agriculture.
Tilley, J. M. A. and R. A. Terry. 1963. A two-stage technique of the in vitro digestion of forage crops. J. Br. Grassl. Soc. 18:104-111.
   2/4/00 M. S. Allen, Dairy Nutrition and Forage Analysis Lab Michigan State University

### Example 3

CAD/COMT Double Knockouts

Co-expression of dsRNAi constructs for CAD & COMT driven by the OsMADs6 promoter can be achieved in single construct. Transgenic maize events containing such constructs are produced using the transformation protocol set forth in Example 1. The sequences of the OsMADs6 promoter and of the RNAi constructs are as presented, and the analyses set forth above are used to determine lignin content etc. Cobs with decreased lignin content produced using this method can be used to the same extent and for the same purposes as those produced using the plasmids of FIG. 1 or 2.

### Example 4

Low Lignin Plant Material use in Biomass Conversions

One of the limitations of converting biomass to ethanol is the need for a harsh chemical pretreatment to separate plant fibers which are “glued” together by lignin. The intent of this invention is that the lower the lignin content, the easier fibers can be separated, less harsh pretreatment used, less lose of glucose during the pretreatment, less enzyme required to hydrolyze the biomass and a higher ethanol yield. An example of the technology using corn cob is presented, however it is obvious that this result can be extended to other plant sources. The production of ethanol from cellulose biomass is discussed in Badger, P. C., Ethanol from cellulose: A general review. p. 17-21, in: J. Janick and A Whipkey (eds.), Trends in new crops and new uses. ASHS Press, Alexandria, Va., 2002.

Standard Pretreatment-saccharification-fermentation

Eight grams of finely ground cob are suspended with 80 ml of a 1% sodium hydroxide solution and heated for 1 hour at 130° C. The pH is then adjusted to pH 5 and 100 milligrams of dry yeast plus 20 filter paper units (FPU) of cellulose is added and allowed to ferment for 20 hours. The resultant beer would be analyzed for ethanol and it would be expected to be around 3% v/v as is usually obtained from fermenting biomass.

Low Lignin Cob Pretreatment-saccharification-fermentation

Eight grams of finely ground cob are suspended with 80 ml of a 1% sodium hydroxide solution and heated for 1 hour at 90° C. The pH is then adjusted to pH 5 and 100 milligrams of dry yeast plus 15 FPU of cellulose is added and allowed to ferment for 20 hours. The resultant beer would be analyzed for ethanol and it would be expected to produce more ethanol than the standard ground cobs in the range of about 3.5% v/v.

### Example 5

Cob Compositional Analysis and Preliminary Hydrolytic Data

Saccharide Compositional Analysis of Corncobs

This example describes the saccharide compositional analysis for glucose, xylose, arabinose, and mannose of corn cobs having low lignin. The major saccharide compositional analysis was determined for three varieties of corncob: CPM913 (Isoline control, genotype A), CPM914 (BM3 mutant, genotype A) and CPM916 (BM3 mutant, genotype B). Composition was determined by performing strong acid hydrolysis (72% H₂SO₄) for one hour, followed by heated dilute-acid hydrolysis (4% H₂SO₄ at 121° C. for 1 hour) and calcium carbonate neutralization. Concentrations of individual saccharide monomers were determined via Refractive Index-High Performance Liquid Chromotography (R1-HPLC). Results are presented in Table 3.

TABLE 3
Compositional analysis of triplicate cob samples. Cob samples were not
analyzed for ferulate, lignin or ash content.

| | glucose | xylose | arabinose | mannose | TOTAL |
|---|---|---|---|---|---|
| | Compositional analysis, Cob samples | | | | |
| CPM 913 | 35% | 21% | 2.9% | 0.3% | 60% |
| CPM 914 | 37% | 23% | 3.2% | 0.3% | 64% |
| CPM 916 | 39% | 23% | 2.9% | 0.1% | 65% |
| | Standard Deviations | | | | |
| CPM 913 | 2.4% | 0.2% | 0.1% | 0.1% | 2.4% |
| CPM 914 | 3.7% | 2.1% | 0.0% | 0.0% | 5.4% |
| CPM 916 | 1.1% | 0.5% | 0.1% | 0.0% | 0.9% |

Enzymatic Hydrolysis of Corn Cobs

This experiment was conducted to determine the saccharides produced from various corn cobs upon enzymatic hydrolysis.

A first-pass screen was initiated using high concentrations of corn cob. CPM914 and CPM916 are reduced lignin genotypes A and B, respectively. Large reactions (100 mg of shredded corn cob) were preferred due to the heterogeneous and course nature of the substrate—generally the reactions took place individual eppendorf tubes rather than microtiter plates. Enzyme extracts from fungal supernatants and a cocktail optimized on corn fiber were tested for hydrolysis activities on the three types of cob. Reactions were 1 ml scale containing a) 100 mg shredded cob, supplemented with b) 50 ug fungal enzymes from Cochliobolus heterotrophus (‘cokie’), c) 50 ug cokie enzymes and 200 ug Aspergillus niger enzymes, d) a xylanase-esterase cocktail containing 2 xylanases, an α-arabinofuranosidase, a β-xylosidase, a ferulic acid esterase and an acetyl xylan esterase. The xylanase cocktail contained: 25 ug BD13509, 125 ug BD2157, 62.5 ug BD13715 and BD13457. The esterase cocktail contained: 100 ug BD14441 and BD14104.

The results are presented in Table 4. When using the defined enzyme cocktail, hydrolysis was higher in both reduced-lignin cob varieties as compared to the regular variety cob. Although the xylanase-esterase cocktail was not optimized for cob hydrolysis, the cocktail had a surprisingly high activity on cob, with better activity on the lower lignin mutants. Note that there were no cellulose-degrading enzymes present in the enzyme cocktail: were these enzymes added, it may be possible to further increase the enzymatic hydrolysis of the cobs.

TABLE 4
Enzymatic hydrolysis of three varieties of shredded corn cob to sugar
monomer, expressed as a percent dry weight.

| | 48 hour timepoint, 10% fiber loading | | | | |
|---|---|---|---|---|---|
| | glucobiose | glucose | xylose | arabinose | TOTAL |
| CPM913 control | 0% | 2.1% | 0% | 0% | 2.1% |
| fiber-induced cokie | 0% | 3.7% | 0.9% | 0.1% | 4.7% |
| cokie + 200 ug fungal enz. | 0.1% | 9.3% | 7.7% | 0.5% | 17.6% |
| xylanase-esterase cocktail | 1.0% | 2.9% | 5.7% | 0% | 9.6% |
| Available sugar CPM 913 | | 35.5% | 20.9% | 2.9% | 59.8% |
| CPM914 control | 0.3% | 2.9% | 0% | 0% | 3.2% |
| fiber-induced cokie | 0.1% | 3.1% | 0.5% | 0% | 3.7% |
| cokie + 200 ug fungal enz. | 0% | 7.1% | 8.5% | 0.7% | 16.3% |
| xylanase-esterase cocktail | 1.2% | 3.1% | 8.5% | 0% | 12.8% |
| Available sugar CPM 914 | | 37.0% | 23.4% | 3.2% | 64.1% |
| CPM916 control | 0% | 2.9% | 0% | 0% | 2.9% |
| fiber-induced cokie | 0.2% | 3.2% | 0.5% | 0% | 3.9% |
| cokie + 200 ug fungal enz. | 0.1% | 7.9% | 10.8% | 0% | 18.9% |
| xylanase-esterase cocktail | 0.8% | 2.4% | 6.8% | 0.2% | 10.2% |
| Available sugar CPM 916 | | 38.8% | 22.8% | 2.9% | 64.8% |

xylanase cocktail: 25 ug BD13509, 125 ug BD2157, 62.5 ug BD13715 and BD13457
esterase cocktail: 100 ug BD14441 and BD14104
Cokie enzymes added at 50 ug

Enzymatic Hydrolysis of Corn Cobs also Including Glucanases, Cellulases and Glucuronidase

The positive relationship between the defined enzyme cocktails and CPM913/CPM914 was further explored, this time adding other glucanases, cellulases and a glucuronidase. Hydrolysis reactions were 1 ml scale, 25 mg/ml shredded corn cob incubated for 48 hrs at 37° C. on an eppendorf tube shaker. The xylanase-esterase cocktail is the same as in Example 2 (10 ul xylanase cocktail: 4 ug 13509, 20 ug 2157, 10 ug 13715 and 13457). Additional enzymes were added to the cocktail: 10 ug α-Glucuronidase BD12669; 100 ug Trichoderma reesi cellulose cocktail; 10 ug glucanase CPM516; glucanase with the esterase cocktail (10 ul esterase cocktail: 100 ug BD14104 and 100 ug BD14441); or a cocktail containing all of the above (except the α-Glucuronidase). With defined enzyme cocktails, the low lignin corncob substrate is consistently more prone to enzymatic attack. Addition of a cellulose extract increases overall hydrolysis to 21.8%, or 34% of the total available sugar. A similar reaction using corn fiber yields 5.4%, a substrate for which the enzymes have been optimized.

The same trend of better digestibility was seen in the low lignin cob samples (Table 5). Of particular interest was the ability of esterases and cellulases to increase the amount of xylose hydrolyzed in the low lignin cob. A combination of xylanase cocktail and cellulases were able to convert 34% of the available sugar to monomer in the low-lignin cob, compared to only 25% in the wild-type cob.

TABLE 5
Hydrolysis of two types of shredded cob using defined enzyme
cocktails - untreated corn fiber is shown for reference. Numbers are
expressed as a percentage dry weight.

| | | | |
|---|---|---|---|
| Glucose | Xylose | Arabinose | TOTAL |

Shredded cob (isoline control genotype A)

| | | | | |
|---|---|---|---|---|
| Control | 2.8% | 0% | 0% | 2.8% |
| xylanase cocktail | 3.5% | 3.7% | 0.2% | 7.4% |
| xylanase w/esterase | 2.9% | 3.4% | 0.2% | 6.5% |
| X-E cocktail w/a-Glrn | 2.8% | 3.8% | 0.2% | 6.8% |
| xylanase w/cellulase | 11.4% | 3.8% | 0.3% | 15.5% |
| glucanase CPM516 | 2.5% | 0.0% | 1.3% | 3.8% |
| CPM516 w/esterase | 2.4% | 0% | 0% | 2.4% |
| CPM516-X-E w/cellulase | 9.3% | 4.4% | 0.2% | 13.9% |
| Available sugar CPM 913 | 35% | 21% | 3% | 60% |

Shredded low-lignin cob (BM3 mutant genotype A)

| | | | | |
|---|---|---|---|---|
| Control | 3.0% | 0% | 0% | 3.0% |
| xylanase cocktail | 2.6% | 3.8% | 0.2% | 6.6% |
| xylanase w/esterase | 3.1% | 5.7% | 0.2% | 9.0% |
| X-E cocktail w/a-Glrn | 3.2% | 5.6% | 0.3% | 9.0% |
| xylanase w/cellulase | 13.0% | 8.5% | 0.3% | 21.8% |
| glucanase CPM516 | 3.0% | 0% | 0% | 3.0% |
| CPM516 w/esterase | 3.0% | 0% | 0% | 3.0% |
| CPM516-X-E w/cellulase | 13.4% | 8.8% | 0.3% | 22.5% |
| Available sugar CPM 914 | 37% | 23% | 3% | 64% |

Untreated corn fiber (w/20% adherent starch)

| | | | | |
|---|---|---|---|---|
| Control | 2.6% | 0% | 0% | 2.6% |
| xylanase cocktail | 2.7% | 0.2% | 0.4% | 3.3% |
| xylanase w/esterase | 2.9% | 0.2% | 0.3% | 3.4% |
| X-E cocktail w/a-Glrn | 3.0% | 0.3% | 0% | 3.3% |
| xylanase w/cellulase | 4.7% | 0.3% | 0.4% | 5.4% |
| glucanase CPM516 | 2.9% | 0% | 0.4% | 3.2% |
| CPM516 w/esterase | 2.9% | 0% | 0% | 2.9% |
| CPM516-X-E w/cellulase | 4.5% | 0.3% | 0.3% | 5.1% |
| Available sugar CPM 711 | 44% | 19% | 10% | 76% |

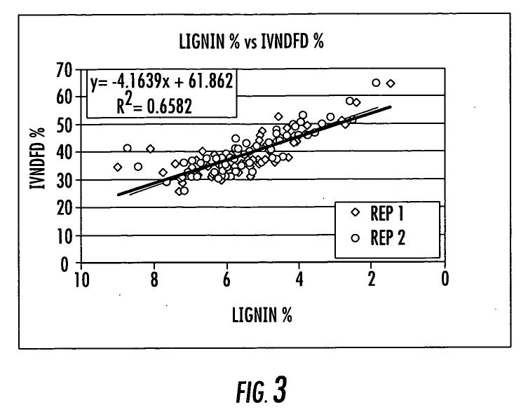

The benefit of improved digestibility of corn silage on dry matter intake and milk yield in dairy cows has been demonstrated by Ballard et al., J. Dairy Sci. 84:442-452 (2001), and by Oba and Allen, J. Dairy Sci. 82:135-142 (1999). Similar feeding trials are established for use of the low-lignin corn cobs of the present invention for demonstration of similar benefits which result from the improved digestibility demonstrated above, and as shown in FIG. 3.

Certain sequences are particularly useful in the practice of the present invention. Those sequences are set forth below:

SEQ ID NO:1. Promoter sequence:OsMADS 6
ctaggacgatggtgtgatgtgggaacacgaagaaaacatgaggaaaaaat
attaaaatgaatttcccacttaaaatgcatcaaataaaaaaaataaagaa
acgaccgggaatagacacagggtttgtgaactagctagggcaaacatcat
atggtcccttgctgatgcacaagtacattgagatgtcatttcaattctgt
gcatcatatgcatgtggtcccttgctgaatattactcttgaaatatctac
cagtgccaatctattgcatgacttaattaattcacaggttttgttgatta
cattattagtaagcttgagagcacaagctcaatggatttttctataaatg
gggatcattttgcaattttctttgtcgtgcaaagttagccttctttatta
ctacttctgtttttaaatatacgatcctattgacttttggtcatatattt
aaccatgtatcttatttagatagtttgcgcaaatatatataccttcaatg
ataaaattagttacaatgaaacaaatgatatttacgcaattctttttact
aaacaagtcacaagaagtacctgcagcaatatatgttggaaccgtgcagt
agatcgagcctagctacgcaaaaaaacaaaaagagaaaaaaagggaaagg
aaaaacattaatcatgcatgagcagtatgcccggcaactggaatttgtca
aagatatggggagaggagaataatacaagtactactactacctagctcta
ccatgcatatgcacccaaaggcaaactggattattggataaagcacagat
gctggcaaaacaatccttaagcctcccctccctgcttctttatttttggg
cagcctctaccggacggtgccgtggtccattggaccagtaggtggcgaca
tacatggtttgggttaagtctaggagagcagtgtgtgtgcgcgcgcaaga
gagagagactgtgagtctgggagtagccctctcccctcctttggccatct
tcctcgtgtatatgcatatatgcatcatcgcaacggtgtatatttgtggt
gtggcgggtgtggcattggattgcccccattttggctcgtgcttcccagt
tagggtaaaacctgtggtaaacttgctagccccacgccaaagttaccctt
ctttattgttgaaagggagaggaggtgtgtgaattgtgatggagggagag
agagagagatagaaagagagatgtgtgtcaaagcaagcaagaaaccagtt
tcacaaagagctactactagtactagtgtactactgtggtacagtgccca
atgtcctttctccggactcgactccactaatattctcctcttctcgcgcg
gctcgttatattctcgtcatcattggaggctttagcaagcaagaagagag
gcagtggtggtggtggtggaggaggagctagctagcctgtgcttgctgat
cggtgctgagctgaggaatcgttcgatcgatcgggcgagtcgacgagggg
aagagttgagctgaggcgcatcgagaacaagatcaacaggcaggtcacct
tctccaagcgccgcaacggcctcctcaagaaggcctacgagctgtccgtt
ctctgcgacgccgaggtcgcgctcatcatcttctccagccgcggcaagct
ctacgagttcggcagcgccgggtataattaatacagacacaacaacacac
acaaccaacaaaccagcatcaatttgaacctgcagatctgctgttttctc
tgatcaattggtagccgagctactatacttacctggccatgttaattatt
ttattccgtctgtctgtgtgtgtctgtgcatactactatagggacatggc
gcggtgttcttataaaccgggaggccggatccctaactagcatgggagga
tatcttttcagcggatctatacaaaccctactcctgctgacctctttctt
ccagtttctccgggtcttccttggattattattgcccatcttccgggttg
tgcgtgtgtcagagacagctcgaacgataaatttctcaaaaccagtacta
gagagggtgtgttgtgtgtgagaactgagtggagagttagcatgaaggct
gcaaactagaaaggaaggtatgttctttcctttttgatccatcaggggag
ccccttctggtattaagatctttccggcacattgattttcatactttgtg
atgaccctggaagaatcggcgtagcagcgtagcaccgctccattttggtc
ttaccctcacctccccatgctatgaactgatcaatttcattgttcttcat
cacccttctcctagctttccacttccttcggatctcatgccatgtttctc
agcatgaatcaaatttaattcgtgttttctacttccatatatactggaag
aaatttaattagatctatttttgctcgggaggtcttcatactttgagttc
tgatgccatcaccttatttcccccccccccttctcttgttctatcttctt
cctcatcttggcttgatcattttgatctgtcagttatagcatgatgcatt
ctcaatttgactgtatgtaagttcaaccggaaatatgttgaatggatttt
ctatatatcaacacttgatgtcaggcctgcatctgtttcgcttgtggtgg
tgtggccaaaattgtctatatttgatctttgctcttctttctcctcattt
catgacgattcctactacggcttaaaccattctttattctttactaatca
tggatgttgcttgactcctagttgtttcgtactagctcaacttggagatc
ttttcattatttgcctagttggtgggtacgtttgtgacagatctaaaatg
gtgcacgaaaagttttacttattatgaaaaaagggagcttaacagggtaa
tttctctatttattcgtgatgacattttttccttgataagggggattttt
tataatctgcactcacatgtttatatgtaaaatctagctcttttgttttg
tttttggcatatttcccgctaagtatagagtttatgtggataacattata
acttttcaagatccaatccacatctttgattgtgaaaatcatacaatagg
gaaaatcaactgaagggttaattagatgctatatgcatatatatatatat
gtgcgcgcgcgcgcgcctgaatttaactatgtatgcatccaactgtttca
ttgaaaaagatttgatatttttcagtctattctttttcgagtatatattt
aatatgtttcaatctgttttgaccattataagataaagcctatattcacc
aggcatttgagatgatcttttcatgcatgaaaaagctgttgttatcactt
caactaaccagacgatctaacatgtatttgtataagaaacagaccttgat
ttccttctgtaaaatcatgcatgtgttcgttttgaattggagtcggcgcg
cctgtgttttgaccgtcaggaaagtcttttttttccctgaatagtcaagg
gtctatacttcttgaagcaattgggacactaatcaattattgtttatacc
tcggaccatcttttccttcttcacaccactaatcagtttatgccttggac
cattaattgtgttgttcacaagcttcttgtttatggtttacaaagcattc
gcctagatttgtgtgtgtctctacacatcgatcacttttaaatacttgtc
gctttcagttattcttttaacgtttggtgcactaatttttcaaataagat
gaatggtcaaatgttacaagaaaaagttaaagcaaccactaatttagggc
ggaggtagtaaaacctagttattgtaaccaataattttatcaatctataa
atgcaacacaaagtcacttcgtgatatctcacacaaagccacttcaacga
tgaaagctgactgcatgttttatcaaaacacatgtgatcagtttgttgga
tgaaaaaaattatctatgtcataaatcaagagttataatataagcttctg
gctctacaagtaacatttctatgttttttttttacgttcttacatactat
gttttgccaaaaaaaacatgatcattttgttggacgaaaagaaatagtaa
atatagagtgacctttgatatcattataatataagcttctgcctctataa
ataacatctatgcactttttacgtcgtagtaatttgatatatgagaaatt
tacatataacatttttgtgcagcataaccacc
SEQ ID NO:2. CAD K/O (pSyn 12210) RNAi sequence
CAD front:
5′atggggagcctggcgtccgagaggaaggtggtcgggtgggccgccagg
gacgccaccggacacctctccccctactcctacaccctcaggaacacagg
ccctgaagatgtggtggtgaaggtgctctactgcgggatctgccacacgg
acatccaccaggccaagaaccacctcggggcttcaaagtatcctatggtc
cctgggcacgaggtggtcggcgaggtggtggaggtcgggcccgaggtggc
caagtacggcgtcggcgacgtggtaggcgtcggggtgatcgttgggtgct
gccgcgagtgcagcccctgcaaggccaacgttgagcagtactgcaacaag
aagatctggtcatacaacgacgtctacactgatggacggcccacgcaggg
tggattcgccctc3′
SEQ ID NO:3. COMT K/O (pSyn 12345) RNAi sequence:
COMT front:
5′atgcagctggcgtcgtcgtccatcctgcccatgacgctgaagaacgcc
atcgagctgggcctgctggaggtgctgcagaaggaggccggcggcggcaa
ggcggcgctggcgcccgaggaggtggtggcgcggatgcccgcggcgcccg
gcgaccccgccgccgcggcggccatggtggaccgcatgctccgcctgctc
gcctcctacgacgtcgtccggtgccagatggaggaccgggacggccggta
cgagcgccgctactccgccgcgcccgtctgcaagtggctcacccccaacg
aggacggcgtgtccatggccgccctcgcgctcatgaaccaggacaaggtc
ctcatggagagctgg3′

Although the foregoing invention has been described in some detail by way of illustration and example for purposes of clarity and understanding, it will be clear to those of skill in the art that certain changes and modifications may be practiced within the scope of the appended claims.

1. A method for controlling lignin biosynthesis in a transformed plant, the method comprising down-regulating the expression of an enzyme in the plant, the enzyme selected from the group consisting of CAD and COMT, wherein the down-regulation is achieved using double-stranded RNAi.

2. The method of claim 1 wherein the plant is corn.

3. The method of claim 2 wherein the down-regulation is localized to the cob of the corn plant.

4. The method of claim 1 wherein the double-stranded RNAi construct comprises SEQ ID NO:2.

5. The method of claim 1 wherein the double-stranded RNAi construct comprises SEQ ID NO:3.

6. A method for controlling lignin biosynthesis in a transformed plant, the method comprising down-regulating the expression of the CAD and COMT genes of a plant using double-stranded RNAi.

7. An RNAi construct selected from the group consisting of the sequence of SEQ ID NO:2 and SEQ ID NO:3.

8. A method for controlling lignin biosynthesis in the cobs of a transformed corn plant, the method comprising down-regulating the expression of the CAD gene, the COMT gene, or both genes, in the cobs of the corn plant using one or more double-stranded RNAi constructs the expression of which is under the control of a cob specific or cob preferred promoter.

9. The method of claim 8 wherein the promoter is the promoter of SEQ ID NO:1.

10. A method for controlling lignin biosynthesis in the cobs of a transformed corn plant, the method comprising down-regulating the expression of the CAD gene, the COMT gene, or both genes, in the cobs of the corn plant by transforming the corn plant with one or more RNAi constructs comprising SEQ ID NO:2, SEQ ID NO:3, or both, wherein the constructs are driven by the promoter of SEQ ID NO:1.

11. A method for the production of ethanol, the method comprising use of biomass comprising material from a low-lignin corn cob produced by the method of claim 8 in an ethanol production process.

12. The method of claim 11 wherein the low-lignin corn cob is produced by the method of claim 10.

13. A method for increasing milk production in an animal by feeding the animal a feed comprising material from a low-lignin corn cob produced by the method of claim 8.

14. The method of claim 13 wherein the low-lignin corn cob is produced by the method of claim 10.

15. A method for increasing the nutritional yield of feed to an animal by feeding the animal a feed comprising material from a low-lignin corn cob produced by the method of claim 8.

16. The method of claim 15 wherein the low-lignin corn cob is produced by the method of claim 10.
